(19) United States
(12) Patent Application Publication
(10) Pub. No.: US 2010/0144652 A1
(43) Pub. Date: Jun. 10, 2010

(54) COMPOSITION COMPRISING A GLYCOLYTIC INHIBITOR AND A RING SYSTEM COMPRISING A SULPHAMATE GROUP FOR THE TREATMENT OF CANCER

(75) Inventors: Sarah Louise Claire Tagg, Berkshire (GB); Paul Alexander Foster, Berkshire (GB); Simon Paul Newman, Berkshire (GB); John Michael Reed, Berkshire (GB); Atul Purohit, Berkshire (GB); Barry Victor Lloyd Potter, Berkshire (GB)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: Sterix Limited, Berkshire (GB)

(21) Appl. No.: 12/526,398

(22) PCT Filed: Feb. 7, 2008
(86) PCT No.: PCT/GB08/00448

§ 371 Date: Jan. 25, 2010

(30) Foreign Application Priority Data
Feb. 8, 2007 (GB) 0702446.6

(51) Int. Cl. A61K 31/7004; A61K 31/70; A61P 35/00

(52) U.S. Cl. 514/23

(57) ABSTRACT

The present invention provides a composition comprising (a) a glycolytic inhibitor (b) a compound comprising a ring system substituted with at least one of a suiphamate group and an alkoxy group; wherein (a) and (b) are different.

### FIELD OF INVENTION

The present invention relates to a composition.

In particular the present invention relates to a composition and to the sequential use of the components of the composition. The present invention also relates to the use of the composition or components thereof in therapy applications.

### BACKGROUND TO THE INVENTION

In 2004, breast and prostate cancer were the third and fourth most common form of cancer, respectively, in Europe (Boyle & Ferlay, 2005). Current treatments for advanced hormone-independent cancer are limited, with resistance and toxicity being common problems with many therapies (Gottesman et al., 2002). There is therefore, a requirement to develop more efficacious therapeutic interventions for treating cancer. Solid tumours are particularly difficult to treat because most conventional chemotherapeutic drugs and radiation therapy only target the rapidly growing peripheral cells, leaving the slower growing tumour cells in the core to survive (Brown & Giaccia, 1998).

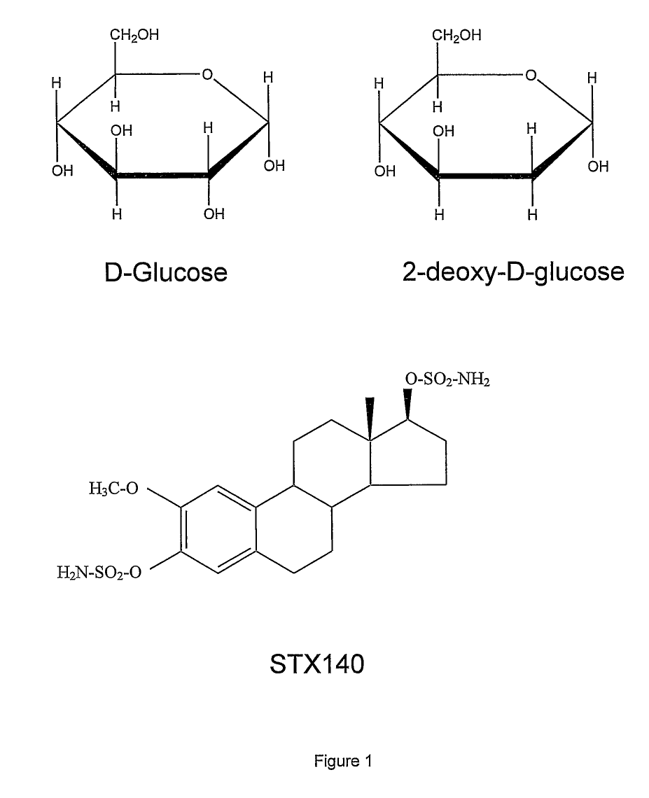

Normal, healthy cells undergo aerobic respiration to produce ATP, however, Warburg first demonstrated that even in the presence of oxygen, cancer cells create ATP anaerobically, relying on glucose for glycolysis (Warburg). Hence, inhibiting glycolysis could specifically target cancer cells; the effects on healthy normal cells should be minimal. Blocking glycolysis is possible by using 2-deoxy-D-glucose (2DG), which is structurally related to glucose and therefore, should not be toxic to healthy cells. The glucose molecule has a hydroxy group at its second carbon, however if this is changed to hydrogen, 2-deoxy-D-glucose (2DG) is formed (see FIG. 1). 2DG blocks glycolysis because when phosphorylated it cannot be converted to fructose-6-phosphate by phosphoglucose isomerase (Nirenberg et al., 1958), in contrast to glucose. In addition, 2DG competes with glucose for uptake by the hexose (GLUT) transporters (Kalir et al., 2002; Noguchi et al., 1999; Rudlowski et al., 2003).

2DG was shown to inhibit growth of adramycin-resistant MCF-7 cells which exhibit an enhanced rate of glycolysis (Kaplan et al., 1990). Several groups have shown 2DG to be an effective anti-tumour compound in vitro and in vivo (Aft et al., 2002; Gupta et al., 2005; Lampidis et al., 2006; Liu et al., 2001; Maschek et al., 2004). However, very few clinical studies have tested 2DG as a single agent therapy and the results of such studies have been disappointing (Landau BR et al., 1958, Kaplin O et al., 1990). In vivo studies have provided strong evidence that using 2DG in combination with existing anti-cancer agents, such as adriamycin or paclitaxel may enhance efficacy (Maschek G et al., 2004). For example, 2DG is currently being used in a Phase 1 trial in combination with the microtubule disruptor Docetaxel in patients with solid tumours (Raez et al., 2005). The microtubule disruptor Docetaxel would attack the rapidly growing cells and the inner core, reliant on glycolysis, could be targeted with 2DG. However, taxanes are toxic, not orally bioavailable and tumours often develop resistance to the taxanes (Polizzi et al, 1999).

### SUMMARY ASPECTS OF THE PRESENT INVENTION

The present invention is based on the surprising finding that action against tumours of a combination of a glycolytic inhibitor and a compound comprising a ring system substituted with at least one of a sulphamate group and an alkoxy group is improved compared to the action of the materials alone or compared to what would be expected from the combination.

### DETAILED ASPECTS OF THE PRESENT INVENTION

According to one aspect of the present invention, there is provided a composition comprising (a) a glycolytic inhibitor (b) a compound comprising a ring system substituted with at least one of a sulphamate group and an alkoxy group; wherein (a) and (b) are different.

According to one aspect of the present invention, there is provided a product comprising (a) a glycolytic inhibitor (b) a compound comprising a ring system substituted with at least one of a sulphamate group and an alkoxy group; wherein (a) and (b) are different, for simultaneous, separate or sequential use in the treatment of cancer.

According to one aspect of the present invention, there is provided a pharmaceutical composition comprising (a) a glycolytic inhibitor (b) a compound comprising a ring system substituted with at least one of a sulphamate group and an alkoxy group; wherein (a) and (b) are different, and a pharmaceutically acceptable carrier, diluent, excipient or adjuvant.

According to one aspect of the present invention, there is provided a composition as defined herein, for use in medicine.

According to one aspect of the present invention, there is provided use of a composition as defined herein, in the manufacture of a medicament to prevent and/or inhibit tumour growth.

According to one aspect of the present invention, there is provided a composition as defined herein for preventing and/or inhibiting tumour growth.

According to one aspect of the present invention, there is provided use of a composition as defined herein, in the manufacture of a medicament for use in the therapy of a condition or disease associated with one or more of steroid sulphatase (STS) activity; cell cycling; apoptosis; cell growth; glucose uptake by a tumour; tumour angiogenesis; microtubules formation; and apoptosis.

According to one aspect of the present invention, there is provided a composition as defined herein, for treatment of a condition or disease associated with one or more of steroid sulphatase (STS) activity; cell cycling; apoptosis; cell growth; glucose uptake by a tumour; tumour angiogenesis; microtubules formation; and apoptosis.

According to one aspect of the present invention, there is provided use of a composition as defined herein, in the manufacture of a medicament for use in the therapy of a condition or disease associated with adverse levels of one or more of steroid sulphatase (STS) activity; cell cycling; apoptosis; cell growth; glucose uptake by a tumour; tumour angiogenesis; microtubules formation; and apoptosis.

According to one aspect of the present invention, there is provided a composition as defined herein for the treatment of a condition or a disease associated with adverse levels of one or more of steroid sulphatase (STS) activity; cell cycling; apoptosis; cell growth; glucose uptake by a tumour; tumour angiogenesis; microtubules formation; and apoptosis.

According to one aspect of the present invention, there is provided use of a composition as defined herein, in the manufacture of a medicament for one or more of inhibiting steroid sulphatase (STS) activity; modulating cell cycling; modulating apoptosis; modulating cell growth; preventing and/or suppressing glucose uptake by a tumour; preventing and/or inhibiting tumour angiogenesis; disrupting microtubules; and inducing apoptosis.

According to one aspect of the present invention, there is provided a composition as defined herein for one or more of inhibiting steroid sulphatase (STS) activity; modulating cell cycling; modulating apoptosis; modulating cell growth; preventing and/or suppressing glucose uptake by a tumour; preventing and/or inhibiting tumour angiogenesis; disrupting microtubules; and inducing apoptosis.

According to one aspect of the present invention, there is provided use of a composition as defined herein, in the manufacture of a medicament for modulating cell growth.

According to one aspect of the present invention, there is provided a composition as defined herein, for modulating cell growth.

According to one aspect of the present invention, there is provided use of a composition as defined herein, in the manufacture of a medicament for treating a cancer.

According to one aspect of the present invention, there is provided a composition as defined herein, for the treatment of cancer.

The cancer may be any susceptible cancer. The cancer may be in the form of a solid tumour. The cancer may in one aspect be breast cancer, ovarian cancer, non-small lung cancer, endometrial cancer, haematological malignancy or prostate cancer. The cancer may in one aspect be breast cancer, ovarian cancer or prostate cancer.

According to one aspect of the present invention, there is provided use of a compound comprising a ring system substituted with at least one of a sulphamate group and an alkoxy group, in the manufacture of a medicament for rendering a tumour susceptible to action by a glycolytic inhibitor.

According to one aspect of the present invention, there is provided a compound comprising a ring system substituted with at least one of a sulphamate group and an alkoxy group for rendering a tumour susceptible to action by a glycolytic inhibitor.

According to one aspect of the present invention, there is provided use of a compound comprising a ring system substituted with at least one of a sulphamate group and an alkoxy group, in the manufacture of a medicament to intensify at least one of hypoxia and glycolysis in a tumour.

According to one aspect of the present invention, there is provided a compound comprising a ring system substituted with at least one of a sulphamate group and an alkoxy group to intensify at least one of hypoxia and glycolysis in a tumour.

According to one aspect of the present invention, there is provided use of a composition as defined herein, in the manufacture of a medicament for decreasing Adenosine 5′-triphosphate (ATP) levels in a tumour.

According to one aspect of the present invention, there is provided a composition as defined herein, for decreasing Adenosine 5′-triphosphate (ATP) levels in a tumour.

According to one aspect of the present invention, there is provided a method of treatment comprising administering to a subject in need of treatment a composition as defined herein.

According to one aspect of the present invention, there is provided a method of treatment comprising administering to a subject in need of treatment a composition as defined herein, in order to inhibit steroid sulphatase (STS) activity; modulate cell cycling; modulate apoptosis; modulate cell growth; prevent and/or suppress glucose uptake by a tumour; prevent and/or inhibit tumour angiogenesis; disrupt microtubules; and/or induce apoptosis.

For ease of reference, these and further aspects of the present invention are now discussed under appropriate section headings. However, the teachings under each section are not necessarily limited to each particular section.

Some Advantages

One key advantage of the present invention is that the compositions of the present invention can prevent and/or inhibit tumour angiogenesis.

One key advantage of the present invention is that the compositions of the present invention can modulate cell cycling.

One key advantage of the present invention is that the compositions of the present invention can modulate apoptosis.

One key advantage of the present invention is that the compositions of the present invention can modulate cell growth.

One key advantage of the present invention is that the compositions of the present invention can prevent and/or suppress glucose uptake by a tumour.

One key advantage of the present invention is that the compositions of the present invention can disrupt microtubules.

One key advantage of the present invention is that the compositions of the present invention can induce apoptosis.

The present invention is based on the surprising finding that the compositions provide an effective treatment of cancer.

Another advantage of the compositions of the present invention is that they may be potent in vivo.

Some of the compositions of the present invention are also advantageous in that they may be orally active.

The compositions of the present invention may useful for the prevention and/or treatment of cancer, such as breast cancer, as well as (or in the alternative) non-malignant conditions, such as the prevention and/or treatment of inflammatory conditions—such as conditions associated with any one or more of: autoimmunity, including for example, rheumatoid arthritis, type I and II diabetes, systemic lupus erythematosus, multiple sclerosis, myasthenia gravis, thyroiditis, vasculitis, endometriosis, ulcerative colitis and Crohn's disease, skin disorders e.g. acne, psoriasis and contact dermatitis; graft versus host disease; eczema; asthma and organ rejection following transplantation. In one aspect the compositions of the present invention may useful for the prevention and/or treatment of endometriosis. The compounds of the present invention are useful particularly when pharmaceuticals may need to be administered from an early age.

In one embodiment, the compounds of the present invention are useful for the treatment of breast cancer.

In one embodiment, the compounds of the present invention are useful for the treatment of prostate cancer.

In one embodiment, the compounds of the present invention are useful for the treatment of ovarian cancer.

Thus, some of the compounds of the present invention are also believed to have therapeutic uses other than for the treatment of endocrine-dependent cancers, such as the treatment of autoimmune diseases.

We have studied combinations of glycolytic inhibitor (such as 2-deoxy-D-glucose [2DG]) and compounds comprising a ring system substituted with at least one of a sulphamate group (such as STX140) in MCF-7 and LNCaP cells in vitro. A MCF-7 (ER+ve) breast xenograft model was used to examine the potency of the composition on the growth of solid tumours. We have found that the present composition disrupt the rapidly dividing aerobic cells and offer a method of targeting both the hypoxic and aerobic cells in tumours.

We have also studied combinations of glycolytic inhibitor (such as 2-deoxy-D-glucose [2DG]) and compounds comprising a ring system substituted with at least one of a sulphamate group (such as STX140) in MCF-7 and LNCaP cells in vivo. We have found that the present composition significantly reduced tumour growth. We have found that the present composition significantly reduced tumour growth. Moreover, use of the present composition may provide for lower doses to be used of compounds comprising a ring system substituted with at least one of a sulphamate group.

For ease of reference, these and further aspects of the present invention are now discussed under appropriate section headings. However, the teachings under each section are not necessarily limited to each particular section.

Preferable Aspects

Glycolytic Inhibtor

As discussed herein, the present invention provides a composition comprising a glycolytic inhibitor. It will be understood that by glycolytic inhibitor it is meant an inhibitor of glycolytic ATP production.

In one preferred aspect the glycolytic inhibitor is a glucose analogue or a glucose conjugate. The terms glucose analogue and glucose conjugate are well understood by one skilled in the art. It will be understood that a glucose analogue mimics glucose while not being metabolised. It will be understood that a glucose conjugate includes materials such as glucose sulphate and glucuronide. In one aspect, the glucose conjugate may be an analogue of a glucose conjugate, such as a glucose sulphamate.

In one preferred aspect the glycolytic inhibitor is a compound of the formula

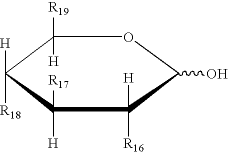

wherein each of R₁₆, R₁₇, R₁₈ is independently selected from H, OH, OSO₂NH₂, OSO₃H, SO₃H, oxamate and halogen and wherein R₁₉ is CH₂OH.

In one preferred aspect the glycolytic inhibitor is a compound of the formula

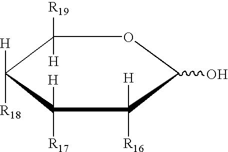

wherein each of R₁₆, R₁₇, R₁₈ is independently selected from H, OH, OSO₂NH₂, OSO₃H, SO₃H, oxamate, and halogen and wherein R₁₉ is CH₃.

Preferably R₁₆ is H, R₁₇ is OH, and R₁₈ is OH.

Highly preferred glycolytic inhibitors for use in the present invention may be selected from 2-deoxy-D-glucose, 1,6-dichloro-1,6-dideoxy-2-deoxyglucose, 2-[N-(7-nitrobenz-2-oxa-1,3-diaxol-4-yl)amino]-2-deoxyglucose (2-NBDG), 2-fluor-2-deoxy-D-glucose (2FG), 2-deoxy-D-galactose, and 3H-2-deoxyglucose.

A highly preferred glycolytic inhibitor for use in the present invention is 2-deoxy-D-glucose.

Compound

As described above the present invention provides a composition or product comprising a compound comprising a ring system substituted with at least one of a sulphamate group and an alkoxy group.

In one preferred aspect the compound comprises a ring system substituted with a sulphamate group and an alkoxy group.

In one preferred aspect the ring system is a steroidal ring system.

Preferably the compound comprises a steroidal ring system ring system substituted with a sulphamate group and an alkoxy group.

In one preferred aspect the compound is of Formula I

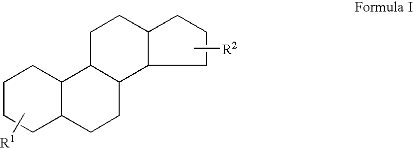

wherein R¹ is selected from —OH and a sulphamate group ; wherein R² is selected from —OH, a sulphamate group, ═O and—L—R³, wherein L is an optional linker group and R³ is selected from groups which are or which comprise one of (i) —SO₂R⁵, wherein R⁵ is H, a hydrocarbyl group or a bond or group attached to the D ring, (ii) —NO₂, (iii) —SOR⁶, wherein R⁶ is H or a hydrocarbyl group, (iv) —R⁷, wherein R⁷ is a halogen, (v) -alkyl, (vi) —C(═O)R⁸, wherein R⁸ is H or hydrocarbyl, (vii) —C≡CR⁹, wherein R⁹ is H or hydrocarbyl, (viii) —OC(═O)NR¹⁰R¹¹, (wherein R¹⁰ and R¹¹ are independently selected from H and hydrocarbyl,

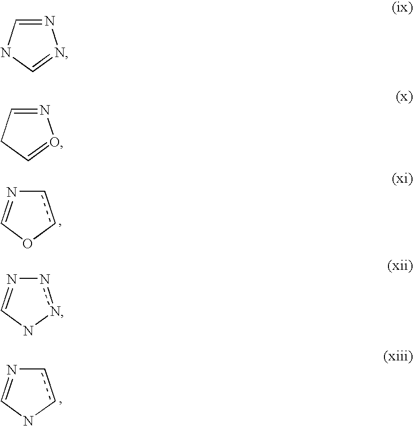

(xiv) a nitrile group, (xv) an alcohol, (xvi) an ester, (xvii) an ether, (xviii) an amine and (xix) an alkene.

The compounds of the present invention may comprise other substituents. These other substituents may, for example, further increase the activity of the compounds of the present invention and/or increase stability (ex vivo and/or in vivo).

Steroidal Ring System

The compound of the present invention has a steroidal ring component—that is to say a cyclopentanophenanthrene skeleton, or bio-isosteres thereof.

As is well known in the art, a classical steroidal ring structure has the generic formula of:

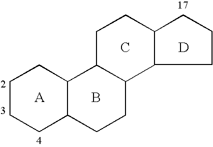

In the above formula, the rings have been labelled and numbered in the conventional manner.

In one aspect, the steroidal ring structure may contain any one or more of C, H, O, N, P, halogen (including Cl, Br and I), S and P.

At least one of the cyclic groups of the steroidal ring structure may be a heterocyclic group (a heterocycle) or a non-heterocyclic group.

At least one of the cyclic groups of the steroidal ring structure may be a saturated ring structure or an unsaturated ring structure (such as an aryl group).

Preferably, at least one of the cyclic groups of the steroidal ring structure is an aryl ring.

An example of a bio-isostere is when any one or more of rings A, B, C and D is a heterocyclic ring and/or when any one or more of rings A, B, C and D has been substituted and/or when any one or more of rings A, B, C and D has been modified; but wherein the bio-isostere has steroidal properties.

In this regard, the structure of a preferred steroidal ring structure can be presented as:

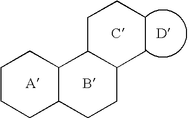

wherein each ring A′, B′, C′ and D′ independently represents a heterocyclic ring or a non-heterocyclic ring, which rings may be independently substituted or unsubstituted, saturated or unsaturated.

By way of example, any one or more of rings A′, B′, C′ and D′ may be independently substituted with suitable groups—such as an alkyl group, an allyl group, an hydroxy group, a halo group, a hydrocarbyl group, an oxyhydrocarbyl group etc.

The term “hydrocarbyl group” as used herein means a group comprising at least C and H and may optionally comprise one or more other suitable substituents. Examples of such substituents may include halo-, alkoxy-, nitro-, a hydrocarbon group, an N-acyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen.

In one preferred embodiment of the present invention, the hydrocarbyl group is a hydrocarbon group.

Here the term “hydrocarbon” means any one of an alkyl group, an alkenyl group, an alkynyl group, an acyl group, which groups may be linear, branched or cyclic, or an aryl group. The term hydrocarbon also includes those groups but wherein they have been optionally substituted. If the hydrocarbon is a branched structure having substituent(s) thereon, then the substitution may be on either the hydrocarbon backbone or on the branch; alternatively the substitutions may be on the hydrocarbon backbone and on the branch.

In one preferred embodiment of the present invention, the hydrocarbyl group is an oxyhydrocarbyl group.

The term “oxyhydrocarbyl group” as used herein means a group comprising at least C, H and O and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the oxyhydrocarbyl group comprises more than one C then those carbons need not necessarily be linked to each other. For example, at least two of the carbons may be linked via a suitable element or group. Thus, the oxyhydrocarbyl group may contain hetero atoms. Suitable hetero atoms will be apparent to those skilled in the art and include, for instance, sulphur and nitrogen.

In one preferred embodiment of the present invention, the oxyhydrocarbyl group is an oxyhydrocarbon group.

Here the term “oxyhydrocarbon” means any one of an alkoxy group, an oxyalkenyl group, an oxyalkynyl group, which groups may be linear, branched or cyclic, or an oxyaryl group. The term oxyhydrocarbon also includes those groups but wherein they have been optionally substituted. If the oxyhydrocarbon is a branched structure having substituent(s) thereon, then the substitution may be on either the hydrocarbon backbone or on the branch; alternatively the substitutions may be on the hydrocarbon backbone and on the branch.

Preferably the oxyhydrocarbyl group is an alkoxy group. Preferably the oxyhydrocarbyl group is of the formula C₁₋₆O (such as a C₁₋₃O).

An example of D′ is a five or six membered non-heterocyclic ring having at least one substituent.

In one preferred embodiment, the ring D′ is substituted with an ethinyl group.

If any one of rings A′, B′, C′ and D′ is a heterocyclic ring, then preferably that heterocyclic ring comprises a combination of C atoms and at least one N atom and/or at least one O atom. Other heterocyclic atoms may be present in the ring.

Examples of suitable, preferred steroidal nuclei rings A′-D′ of the compounds of the present invention include rings A-D of oestrone and dehydroepiandrosterone.

Preferred steroidal nuclei rings A′-D′ of the compounds of the present invention include rings A-D of:

oestrones and substituted oestrones, viz:

oestrone

2-OH-oestrone

2-alkoxy-oestrone (such as C₁₋₆ alkoxy-oestrone, such as 2-methoxy-oestrone)

4-OH-oestrone

6α-OH-oestrone

7α-OH-oestrone

16α-OH-oestrone

16β-OH-oestrone

oestradiols and substituted oestradiols, viz:

2-OH-17β-oestradiol

2-alkoxy-17β-oestradiol (such as C₁₋₆ alkoxy-17β-oestradiol, such as 2-methoxy-17β-oestradiol)

4-OH-17β-oestradiol

6α-OH-17β-oestradiol

7α-OH-17β-oestradiol

2-OH-17α-oestradiol

2-alkoxy-17α-oestradiol (such as C₁₋₆ alkoxy-17α-oestradiol, such as 2-methoxy-17α-oestradiol)

4-OH-17α-oestradiol

7α-OH-17α-oestradiol

16α-OH-17α-oestradiol

16α-OH-17α-oestradiol

16α-OH-17β-oestradiol

16β-OH-17α-oestradiol

16β-OH-17β-oestradiol

17α-oestradiol

17β-oestradiol

17α-ethinyl-17β-oestradiol

17β-ethinyl-17α-oestradiol oestriols and substituted oestriols, viz:

oestriol

2-OH-oestriol

2-alkoxy-oestriol (such as C₁₋₆ alkoxy-oestriol, such as 2-methoxy-oestriol)

4-OH-oestriol

6α-OH-oestriol

7α-OH-oestriol

dehydroepiandrosterones and substituted dehydroepiandrosterones, viz:

dehydroepiandrosterones

6α-OH-dehydroepiandrosterone

7α-OH-dehydroepiandrosterone

16α-OH-dehydroepiandrosterone

16β-OH-dehydroepiandrosterone

In general terms the ring system A′B′C′D′ may contain a variety of non-interfering substituents. In particular, the ring system A′B′C′D′ may contain one or more hydroxy, alkyl especially lower (C₁-C₆) alkyl, e.g. methyl, ethyl, n-propyl, isopropyl, n-butyl, sec-butyl, tert-butyl, n-pentyl and other pentyl isomers, and n-hexyl and other hexyl isomers, alkoxy especially lower (C₁-C₆) alkoxy, e.g. methoxy, ethoxy, propoxy etc., alkinyl, e.g. ethinyl, or halogen, e.g. fluoro substituents.

In an alternative embodiment, the polycyclic compound may not contain or be based on a steroid nucleus. In this regard, the polycyclic compound may contain or be based on a non-steroidal ring system—such as diethylstilboestrol, stilboestrol, coumarins, and other ring systems. Other suitable non-steroidal compounds for use in or as the composition of the present invention may be found in U.S. Pat. No. 5,567,831.

R¹ and R²

In one preferred aspect the compound is of Formula I

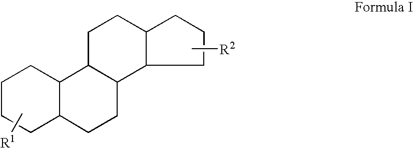

In one preferred aspect the compound is of Formula Ia

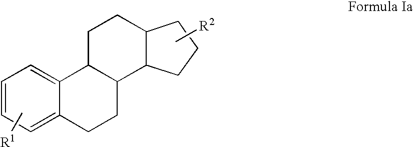

In one preferred aspect the compound is of Formula Ib

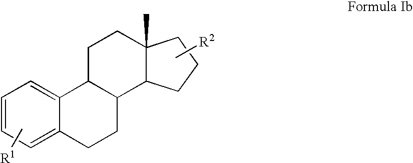

In one preferred aspect the compound is of Formula II

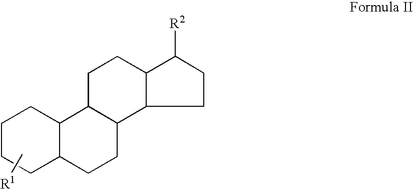

In one preferred aspect the compound is of Formula IIa

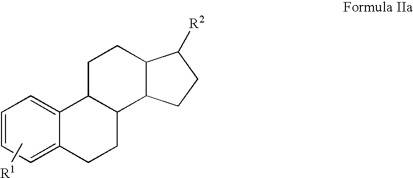

In one preferred aspect the compound is of Formula IIb

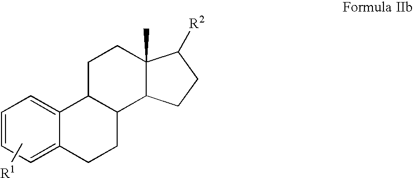

In one preferred aspect the compound is of Formula III

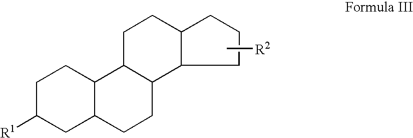

In one preferred aspect the compound is of Formula IIIa

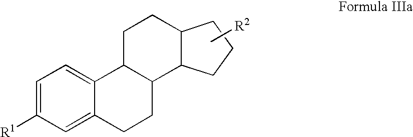

In one preferred aspect the compound is of Formula IIIb

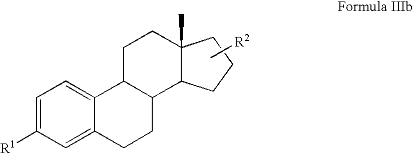

In one preferred aspect the compound is of Formula IVa or Formula IVb (preferably of Formula IVa)

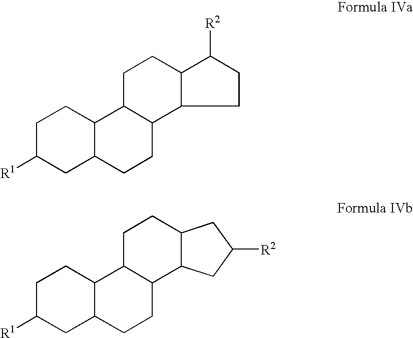

In one preferred aspect the compound is of Formula IVc or Formula IVd (preferably of Formula IVc)

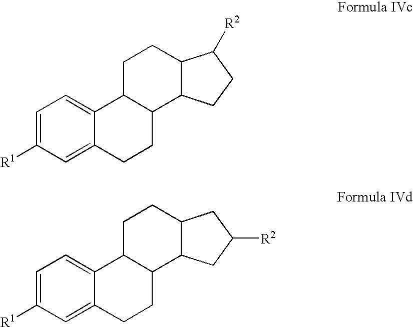

In one preferred aspect the compound is of Formula IVe or Formula IVf (preferably of Formula IVe)

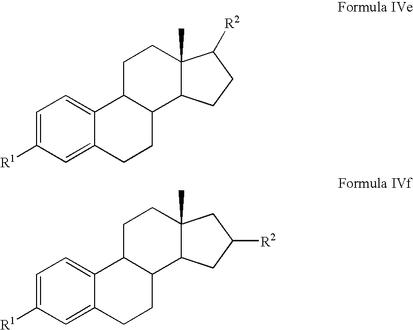

In one preferred aspect the compound is of Formula Va or Formula Vb (preferably of Formula Va)

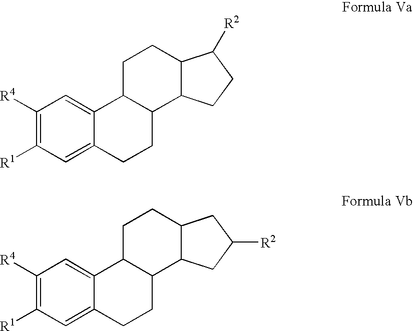

In one preferred aspect the compound is of Formula Vc or Formula Vd (preferably of Formula Vc)

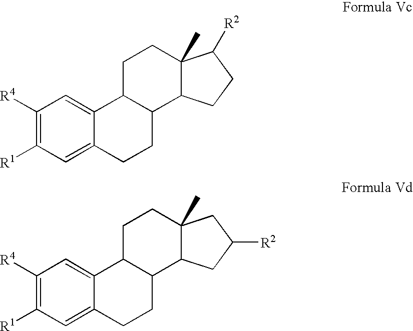

R¹

It will be appreciated by one skilled in the art that R¹ is an optional group which may or may not be present. In one preferred aspect R¹ is present. In this aspect R¹ is a group selected from any one of —OH, a sulphamate group, a phosphonate group, a thiophosphonate group, a sulphonate group or a sulphonamide group.

Sulphamate Group

In one aspect R¹ is an optional sulphamate group.

The term “sulphamate” includes an ester of sulphamic acid, or an ester of an N-substituted derivative of sulphamic acid, or a salt thereof.

In one aspect R¹ is a sulphamate group. In this aspect the compound of the present invention may be referred to as a sulphamate compound.

Preferably the sulphamate group of R¹, is a sulphamate group of the formula

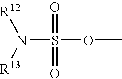

wherein R¹² and R¹³ are independently selected from H or a hydrocarbyl group.

Preferably R¹² and R¹³ are independently selected from H, alkyl, cycloalkyl, alkenyl, acyl and aryl, or combinations thereof, or together represent alkylene, wherein the or each alkyl or cycloalkyl or alkenyl or aryl optionally contains one or more hetero atoms or groups (such as O, S and N).

When substituted, the N-substituted compounds of this invention may contain one or two N-alkyl, N-alkenyl, N-cycloalkyl, N-acyl, or N-aryl substituents, preferably containing or each containing a maximum of 10 carbon atoms. When R¹² and/or R¹³ is alkyl, the preferred values are those where R¹² and R¹³ are each independently selected from lower alkyl groups containing from 1 to 5 carbon atoms, that is to say methyl, ethyl, propyl etc. Preferably R⁵ and R⁶ are both methyl. When R¹² and/or R¹³ is aryl, typical values are phenyl and tolyl (-PhCH₃; o-, m- or p-). Where R⁵ and R⁶ represent cycloalkyl, typical values are cyclopropyl, cyclopentyl, cyclohexyl etc. When joined together R¹² and R¹³ typically represent an alkylene group providing a chain of 4 to 6 carbon atoms, optionally interrupted by one or more hetero atoms or groups, e.g. —OH— or —NH— to provide a 5-, 6- or 7-membered heterocycle, e.g. morpholino, pyrrolidino or piperidino.

Within the values alkyl, cycloalkyl, alkenyl, acyl and aryl we include substituted groups containing as substituents therein one or more groups which do not interfere with the sulphatase inhibitory activity of the compound in question. Exemplary non-interfering substituents include hydroxy, amino, halo, alkoxy, alkyl and aryl. A non-limiting example of a hydrocarbyl group is an acyl group.

In some embodiments, the sulphamate group may form a ring structure by being fused to (or associated with) one or more atoms in or on the steroidal ring system.

In some embodiments, there may be more than one sulphamate group. By way of example, there may be two sulphamates (i.e. bis-sulphamate compounds).

In some preferred embodiments, at least one of R¹² and R¹³ is H.

In some preferred embodiments, each of R¹² and R¹³ is H.

In some preferred embodiments R¹ is a sulphamate group and the compound is suitable for use as an inhibitor of oestrone sulphatase (E.G. 3.1.6.2).

In some preferred embodiments if the sulphamate group on the sulphamate compound were to be replaced with a sulphate group to form a sulphate compound then the sulphate compound would be hydrolysable by a steroid sulphatase enzyme (E.C.3.1.6.2).

In some preferred embodiments if the sulphamate group on the sulphamate compound were to be replaced with a sulphate group to form a sulphate compound and incubated with a steroid sulphatase enzyme (E.C.3.1.6.2) at a pH 7.4 and 37° C. it would provide a Kₘ value of less than 50 mM.

In some preferred embodiments if the sulphamate group on the sulphamate compound were to be replaced with a sulphate group to form a sulphate compound and incubated with a steroid sulphatase enzyme (E.C.3.1.6.2) at a pH 7.4 and 37° C. it would provide a Kₘ value of less than 50 μM.

Other Substituents

The compound of the present invention may have substituents other than those of formula I. By way of example, these other substituents may be one or more of: one or more sulphamate group(s), one or more phosphonate group(s), one or more thiophosphonate group(s), one or more sulphonate group(s), one or more sulphonamide group(s), one or more halo groups, one or more O groups, one or more hydroxy groups, one or more amino groups, one or more sulphur containing group(s), one or more hydrocarbyl group(s)—such as an oxyhydrocarbyl group.

R²

R² is selected from —OH, a sulphamate group, ═O and—L—R³, wherein L is an optional linker group and R³ is selected from groups which are or which comprise one of (i) —SO₂R⁵, wherein R⁵ is H, a hydrocarbyl group or a bond or group attached to the D ring, (ii) —NO₂, —SOR⁶, wherein R⁶ is H or a hydrocarbyl group, (iv) —R⁷, wherein R⁷ is a halogen, (v) -alkyl, (vi) —C(═O)R⁸, wherein R⁸ is H or hydrocarbyl, (vii) —C≡CR⁹, wherein R⁹ is H or hydrocarbyl, (viii) —OC(═O)NR¹⁰R¹¹, wherein R¹⁰ and R¹¹ are independently selected from H and hydrocarbyl,

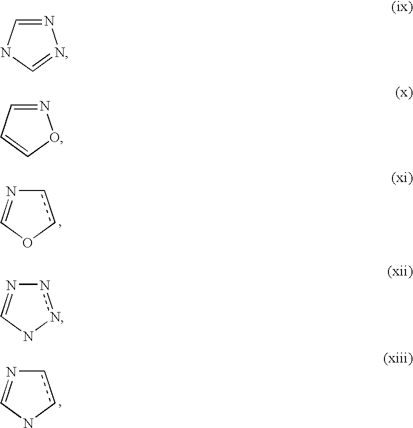

(xiv) a nitrile group, (xv) an alcohol, (xvi) an ester, (xvii) an ether, (xviii) an amine and (xix) an alkene.

In some preferred embodiments R² is of the formula —R³, in other words no group L is present.

In a preferred aspect R² is selected from —OH, a sulphamate group.

In a highly preferred aspect R² is a sulphamate group. Preferably R² is a sulphamate group of the formula

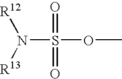

wherein R¹² and R¹³ are independently selected from H, alkyl, cycloalkyl, alkenyl and aryl, or combinations thereof, or together represent alkylene, wherein the or each alkyl or cycloalkyl or alkenyl or aryl optionally contains one or more hetero atoms or groups (such as O, N and S).

When R¹² and/or R¹³ is alkyl, the preferred values are those where R¹² and R¹³ are each independently selected from lower alkyl groups containing from 1 to 5 carbon atoms, that is to say methyl, ethyl, propyl etc. Preferably R¹² and R¹³ are both methyl. When R¹² and/or R¹³ is aryl, typical values are phenyl and tolyl (-PhCH₃; o-, m- or p-). Where R¹² and R¹³ represent cycloalkyl, typical values are cyclopropyl, cyclopentyl, cyclohexyl etc. When joined together R¹² and R¹³ typically represent an alkylene group providing a chain of 4 to 6 carbon atoms, optionally interrupted by one or more hetero atoms or groups, e.g. —O— or —NH— to provide a 5-, 6- or 7-membered heterocycle, e.g. morpholino, pyrrolidino or piperidino.

In one aspect R¹² and R¹³ are each independently hydrocarbyl and H. In one preferred embodiment of the present invention R¹² and R¹³ are each independently selected from one of H, C₁-C₂₀ hydrocarbyl, C₁-C₁₀ hydrocarbyl, C₁-C₅ hydrocarbyl, C₁-C₃ hydrocarbyl, hydrocarbon groups,. C₁-C₂₀ hydrocarbon, C₁-C₁₀ hydrocarbon, C₁-C₅ hydrocarbon, C₁-C₃ hydrocarbon, alkyl groups, C₁-C₂₀ alkyl, C₁-C₁₀ alkyl, C₁-C₅ alkyl, and C₁-C₃ alkyl

In one aspect R¹² and R¹³ are each independently selected from H and C₁₋₁₀ alkyl. In one aspect R¹² and R¹³ are each independently C₁₋₁₀ alkyl. In one aspect R¹² and R¹³ are each independently selected from H and C₁₋₅ alkyl. In one aspect R¹² and R¹³ are each independently C₁₋₅ alkyl. In one aspect R¹² and R¹³ are each independently selected from H and C₁₋₃ alkyl. In one aspect R¹² and R¹³ are each independently C₁₋₃ alkyl. Preferably R¹² and R¹³ are independently selected from —H and —CH₃.

In some preferred embodiments, at least one of R¹² and R¹³ is H.

In some preferred embodiments, each of R¹² and R¹³ is H.

In some preferred aspects group R² is in an a configuration. Preferably group R² is in an a configuration on the 17 position of the D ring.

L

In some embodiments L is selected from a hydrocarbyl group, —NR¹⁴— and —O—, wherein R¹⁴ is H, a hydrocarbyl group or a bond.

Preferably L is selected from a hydrocarbon group, —NR¹⁴— and —O—.

In one aspect L is selected from an alkylene group (such as C₁₋₁₀ alkylene, a C₁₋₅ alkylene, a C₁ or C₂ alkylene), —NR¹⁴— and —O—.

In one aspect L is selected from a C₁₋₁₀ alkylene group, —NR¹⁴— and —O—.

In one aspect L is selected from a C₁ or C₂ alkylene group, —NR¹⁴— and —O—.

Particularly preferred linkers are ═N—, —NH—, ═CH—, —CH₂—, —CH₂CH₂— and ═CHCH₂—, such as ═N—, —NH—, ═CH—, and —CH₂—.

R³

As discussed above R³ is selected from (i) —SO₂R⁵, wherein R⁵ is H, a hydrocarbyl group or a bond or group attached to the D ring, (ii) —NO₂, (iii) —SOR^{B}, wherein R⁶ is H or a hydrocarbyl group, (iv) —R⁷, wherein R⁷ is a halogen, (v) -alkyl, (vi) —C(═O)R⁸, wherein R⁸ is H or hydrocarbyl, (vii) —C═CR⁹, wherein R⁹ is H or hydrocarbyl, (viii) —OC(═O)NR¹⁰R¹¹, wherein R¹⁰ and R¹¹ are independently selected from H and hydrocarbyl,

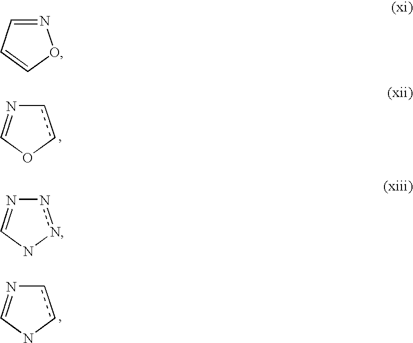

(xiv) a nitrile group, (xv) an alcohol, (xvi) an ester, (xvii) an ether, (xviii) an amine and (xix) an alkene.

R³ may be a cyclic group or an acyclic group.

When R³ is a cyclic group is may form a ring which is fused with the D ring of the steroid or which is not fused with the D ring of the steroid. When R³ forms a cyclic group which is fused with the D ring of the steroid, preferably R³ forms a ring joining adjacent members of the D ring, more preferably R³ forms a ring joining positions 16 and 17 of the D ring.

It will be appreciated by one skilled in the art that group R³ may be attached to optional L at any point on R³. Preferred points of attachment are shown when groups (ix) to (xiiii) are selected from optionally substituted groups of the formulae

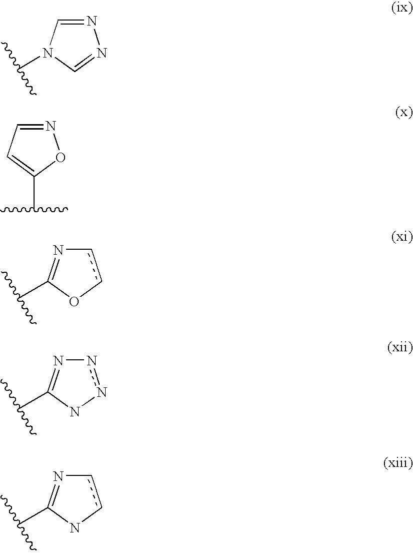

—SO₂R⁵

In one preferred aspect R³ is —SO₂R⁵, wherein R⁵ is H, a hydrocarbyl group or a bond or group attached to the D ring

Preferably R⁵ is selected from H and hydrocarbyl. In one aspect R⁵ is hydrocarbyl. In one preferred embodiment of the present invention R⁵ is selected from one of H, C₁-C₂₀ hydrocarbyl, C₁-C₁₀ hydrocarbyl, C₁-C₅ hydrocarbyl, C₁-C₃ hydrocarbyl, hydrocarbon groups, C₁-C₂₀ hydrocarbon, C₁-C₁₀ hydrocarbon, C₁-C₅ hydrocarbon, C₁-C₃ hydrocarbon, alkyl groups, C₁-C₂₀ alkyl, C₁-C₁₀ alkyl, C₁-C₅ alkyl, and C₁-C₃ alkyl.

In one aspect R⁵ is selected from H and C₁₋₁₀ alkyl. In one aspect R⁵ is C₁₋₁₀ alkyl. In one aspect R⁵ is selected from H and C₁₋₅ alkyl. In one aspect R⁵ is C₁₋₅ alkyl. In one aspect R⁵ is selected from H and C₁₋₃ alkyl. In one aspect R⁵ is C₁₋₃ alkyl. Preferably R⁵ is —CH₃.

Preferably R⁵ is —O—R¹⁵-D, wherein R¹⁵ is a linker and D is a member of the D ring. In a preferred aspect this provides a compound of the formula

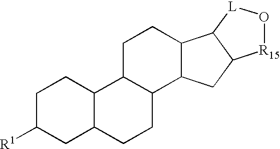

R¹⁵ may be any suitable group. Particularly preferred are —O—CH₂— and —N═CH—

In this aspect preferably R² is —CH₂—R³ or —NH—R³, for example in one preferred aspect R² is —NH—SO₂—CH₃.

—NO₂

In one preferred aspect wherein R³ is —NO₂

In this aspect preferably R² is —CH₂—R³

—SOR⁶

In one preferred aspect R³ is —SOR⁶, wherein R⁶ is H or a hydrocarbyl group.

Preferably R⁶ is selected from H and hydrocarbyl. In one aspect R⁶ is hydrocarbyl. In one preferred embodiment of the present invention R⁶ is selected from one of H, C₁-C₂₀ hydrocarbyl, C₁-C₁₀ hydrocarbyl, C₁-C₅ hydrocarbyl, C₁-C₃ hydrocarbyl, hydrocarbon groups, C₁-C₂₀ hydrocarbon, C₁-C₁₀ hydrocarbon, C₁-C₅ hydrocarbon, C₁-C₃ hydrocarbon, alkyl groups, C₁-C₂₀ alkyl, C₁-C₁₀ alkyl, C₁-C₅ alkyl, and C₁-C₃ alkyl.

In one aspect R⁶ is selected from H and C₁-C₁₀ alkyl. In one aspect R⁶ is C₁-C₁₀ alkyl. In one aspect R⁶ is selected from H and C₁-C₅ alkyl. In one aspect R⁶ is C₁-C₅ alkyl. In one aspect R⁶ is —CH₃. is selected from H and C₁₋₃ alkyl. In one aspect R⁶ is C₁₋₃ alkyl. Preferably R⁶ is —CH₃.

In this aspect preferably R² is —CH₂—R³

R⁷

In one preferred aspect R³ is —R⁷, wherein R⁷ is a halogen

It will be appreciated that R⁷ may chlorine, fluorine, bromine or iodine. Preferably R⁷ is fluorine.

In this aspect preferably R² is —CH₂CH₂—R³, namely —CH₂CH₂—R⁷.

In this aspect preferably R² is —CH₂CHX—R⁷ wherein X is a halogen. For example X may be F and R⁷ may be F such that R² is —CH₂CF₂H.

In this aspect R² may also be —CX₂—R³, wherein each X is independently selected from halogens. For example each X may be F and R³ may be F such that R² is CF₃.

In this aspect R² may be —CY₂—R³ or —CY₂CY₂—R³, wherein each Y is independently selected from H and halogens. For example one or more Y may be F and R³ may be F. When only one Y is H and the remaining Y are H, R² may be —CHY—R³ or —CH₂CHY—R³, wherein Y is selected from H and halogens. For example Y may be F and R³ may be F.

-alkyl

In one preferred aspect R³ is -alkyl

In one preferred embodiment of the present invention R³ is selected from one of C₁-C₂₀ alkyl, C₁-C₁₀ alkyl, C₁-C₅ alkyl, and C₁-C₃ alkyl.

In one aspect R³ is C₁₋₁₀ alkyl. In one aspect R³ is C₁₋₅ alkyl. In one aspect R³ is C₁₋₃ alkyl. Preferably R³ is —CH₃ or —CH₂CH₃.

In this aspect preferably R² is R³.

In one preferred aspect when R³ is alkyl the compound is of Formula IVb

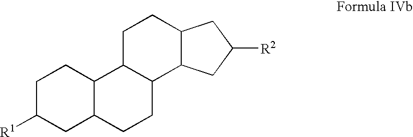

such as of Formula IVd

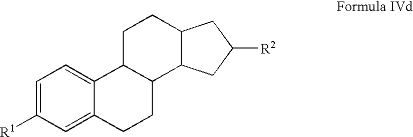

such as of Formula IVf

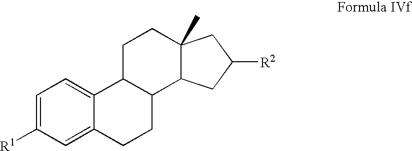

such as of Formula Vb

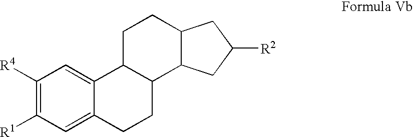

such as of Formula Vd

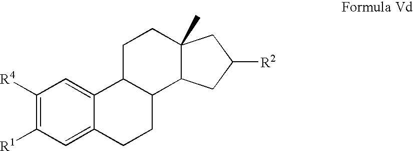

In one aspect the compound further comprises a further group denoted R²′ which is an alkyl group and preferably an alkyl group described under (v) herein. Thus in one preferred aspect the compound is selected from compounds of the formulae

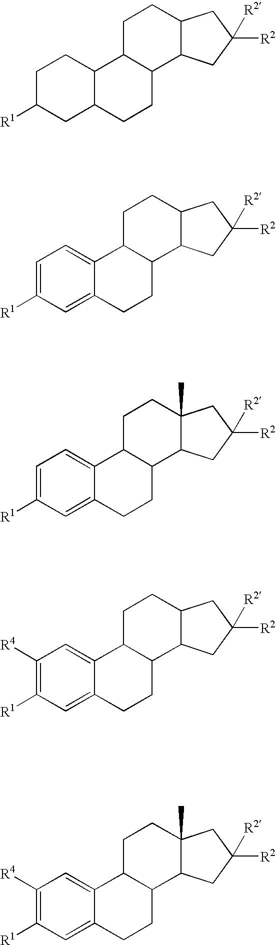

wherein R² and R²′ are independently selected from one of C₁-C₂₀ hydrocarbyl, C₁-C₁₀ hydrocarbyl, C₁-C₅ hydrocarbyl, C₁-C₃ hydrocarbyl, hydrocarbon groups, C₁-C₂₀ hydrocarbon, C₁-C₁₀ hydrocarbon, C₁-C₅ hydrocarbon, C₁-C₃ hydrocarbon, alkyl groups, C₁-C₂₀ alkyl, C₁-C₁₀ alkyl, C₁-C₅ alkyl, and C₁-C₃ alkyl. In a highly preferred aspect each of R² and R²′ are —CH₃.

—C(═O)R⁸

In one preferred aspect R³ is —C(═O)R⁸, wherein R⁸ is H or hydrocarbyl

Preferably R⁸ is selected from H and hydrocarbyl. In one aspect R⁸ is hydrocarbyl. In one preferred embodiment of the present invention R⁸ is selected from one of H, C₁-C₂₀ hydrocarbyl, C₁-C₁₀ hydrocarbyl, C₁-C₅ hydrocarbyl, C₁-C₃ hydrocarbyl, hydrocarbon groups, C₁^{-C}₂₀ hydrocarbon, C₁-C₁₀ hydrocarbon, C₁-C₅ hydrocarbon, C₁-C₃ hydrocarbon, alkyl groups, C₁-C₂₀ alkyl, C₁-C₁₀ alkyl, C₁-C₅ alkyl, and C₁-C₃ alkyl.

In one aspect R⁸ is selected from H and C₁₋₁₀ alkyl. In one aspect R⁸ is C₁₋₁₀ alkyl. In one aspect R⁸ is selected from H and C₁₋₅ alkyl. In one aspect R⁹ is C₁₋₅ alkyl. In one aspect R⁸ is selected from H and C₁₋₃ alkyl. In one aspect R⁸ is C₁₋₃ alkyl. Preferably R⁸ is —CH₃.

In this aspect preferably R² is —CH₂—R³ or R³, for example —C(═O)CH₃.

—C≡CR⁹

In one preferred aspect R³ is —C≡CR⁹, wherein R⁹ is H or hydrocarbyl

Preferably R⁹ is selected from H and hydrocarbyl. In one aspect R⁹ is hydrocarbyl. In one preferred embodiment of the present invention R⁹ is selected from one of H, C₁-C₂₀ hydrocarbyl, C₁-C₁₀ hydrocarbyl, C₁-C₅ hydrocarbyl, C₁-C₃ hydrocarbyl, hydrocarbon groups, C₁-C₂₀ hydrocarbon, C₁-C₁₀ hydrocarbon, C₁-C₅ hydrocarbon, C₁-C₃ hydrocarbon, alkyl groups, C₁-C₂₀ alkyl, C₁-C₁₀ alkyl, C₁-C₅ alkyl, and C₁-C₃ alkyl.

In one aspect R⁹ is selected from H and C₁₋₁₀ alkyl. In one aspect R⁹ is C₁₋₁₀ alkyl. In one aspect R⁹ is selected from H and C₁₋₅ alkyl. In one aspect R⁹ is C_{1-C}₅ alkyl. In one aspect R⁹ is selected from H and C₁₋₃ alkyl. In one aspect R⁹ is C₁₋₃ alkyl. Preferably R⁹ is —CH₃.

In this aspect preferably R² is —CH₂—R³

—OC(═O)NR¹⁰R¹¹

In one preferred aspect R³ is —OC(═O)NR¹⁰R¹¹, wherein R¹⁰ and R¹¹ are independently selected from H and hydrocarbyl

Preferably R¹⁰ and R¹¹ are independently selected from H and hydrocarbyl. In one aspect R¹⁰ and R¹¹ are independently selected from hydrocarbyl. In one preferred embodiment of the present invention R¹⁰ and R¹¹ are independently selected from one of H, C₁-C₂₀ hydrocarbyl, C₁-C₁₀ hydrocarbyl, C₁-C₅ hydrocarbyl, C₁-C₃ hydrocarbyl, hydrocarbon groups, C₁-C₂₀ hydrocarbon, C₁-C₁₀ hydrocarbon, C₁-C₅ hydrocarbon, C₁-C₃ hydrocarbon, alkyl groups, C₁-C₂₀ alkyl, C₁-C₁₀ alkyl, C₁-C₅ alkyl, and C₁-C₃ alkyl.

In one aspect R¹⁰ and R¹¹ are independently selected from H and C₁₋₁₀ alkyl. In one aspect R¹⁰ and R¹¹ are independently selected from C₁₋₁₀ alkyl. In one aspect R¹⁰ and R¹¹ are independently selected from H and C₁₋₅ alkyl. In one aspect R¹⁰ and R¹¹ are independently selected from C₁₋₅ alkyl. In one aspect R¹⁰ and R¹¹ are independently selected from H and C₁₋₃ alkyl. In one aspect R¹⁰ and R¹¹ are independently selected from C₁₋₃ alkyl. Preferably R¹⁰ and R¹¹ are both H.

In this aspect preferably R² is R³.

Cyclic Groups

In one preferred aspect R³ is

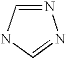

Preferably R³ is

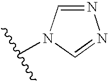

In this aspect preferably R² is selected from —CH₂CH₂—R³, ═N—R³ and —NH—R³

In one preferred aspect wherein R³ is

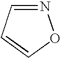

Preferably R³ is

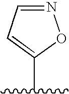

Preferably R³ is

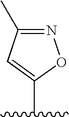

In this aspect preferably R² is selected from ═CH—R³ and —CH₂CH₂—R³

In one preferred aspect wherein R³ is

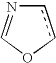

Preferably R³ is

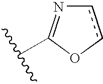

In this aspect preferably R² is selected from ═CH—R³ and —CH₂CH₂—R³

In one preferred aspect R³ is

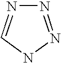

Preferably R³ is

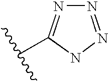

Preferably R³ is selected from

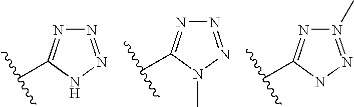

In this aspect preferably R² is selected from ═CH—R³ and —CH₂CH₂—R³

In one preferred aspect R³ is

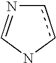

Preferably R³ is

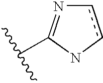

In this aspect preferably R² is selected from ═CH—R³ and —CH₂CH₂—R³

In one preferred aspect R³ is selected from groups which are or which comprise one of a nitrile group, an alcohol, an ester, an ether, an amine and an alkene In some preferred aspects R³ is selected the groups nitrile, alcohol, ester, ether, amine and alkene. Preferably R³ is or comprises a nitrile group. Preferably R³ is a nitrile group.

In some preferred aspects R³ is selected from groups of the formula ═CH₂, ═CH—CH₃, ═C(CN)₂, ═C(CH₃)(CN), and —(R^{7a})ₙ(CR^{14a}R^{15a})^{p}R^{8a}, wherein n is 0 or or 1, p is an integer R^{7a} is selected from ═CH—, —O— and NR^{13a}; R^{8a} is selected from —SO₂—R^{9a}, —C(O)OR^{17a}, —OR^{10a}, (CH₂)_{q}—X—R^{16a}, —C≡N, —NR^{11a}R^{12a}—C≡CH and —CH═CH₂; R^{9a} is selected from H and hydrocarbyl, R^{10a} is selected from H and hydrocarbyl; R^{11a} and R^{12a} are each independently selected from H and hydrocarbyl; R^{13a} is selected from H and hydrocarbyl, R^{14a} and R^{15a} are each independently selected from H and hydrocarbyl, q is an integer, X is O or S, R^{16a} is selected from H and hydrocarbyl and R^{17a} is selected from H and hydrocarbyl.

In some preferred aspects R³ is a group of the formula —(R^{7a})ₙ(CR^{14a}R^{15a})ₚR^{8a}, where n is 0 or 1, p is an integer, R^{7a} is selected from ═CH—, —O— and NR^{13a}; R^{8a} is selected from —SO₂—R^{9a}, —C(O)OR^{17a}, —OR^{10a}, (CH₂)_{q}—X—R^{16a}, —C≡N, —NR^{11a}R^{12a}—C≡CH and —CH═CH₂; R^{9a} is selected from H and hydrocarbyl, R^{10a} is selected from H and hydrocarbyl; R^{11a} and R^{12a} are each independently selected from H and hydrocarbyl; R¹³ is selected from H and hydrocarbyl, R^{14a} and R^{15a} are each independently selected from H and hydrocarbyl, q is an integer, X is O or S, R^{16a} is selected from H and hydrocarbyl and R¹⁷ is selected from H and hydrocarbyl.

In some preferred aspects R³ is a group of the formula —(CR^{14a}R^{15a})ₚR^{8a}, p is an integer; R^{8a} is selected from —SO₂-R^{9a}, —C(O)OR^{17a}, —OR^{10a}, (CH₂)_{q}—X—R^{16a}, —C≡N, —NR^{11a}R¹²—C≡CH and —CH═CH₂; R^{9a} is selected from H and hydrocarbyl, R^{10a} is selected from H and hydrocarbyl; R^{11a} and R^{12a} are each independently selected from H and hydrocarbyl, R^{14a} and R^{15a} are each independently selected from H and hydrocarbyl, q is an integer, X is O or S, R^{16a} is selected from H and hydrocarbyl and R^{17a} is selected from H and hydrocarbyl.

In some preferred aspects R³ is a group of the formula —(CH₂)ₚR^{8a}, p is an integer; R^{8a} is selected from —SO₂—R^{9a}, —C(O)OR^{17a}, —OR^{10a}, (CH₂)_{q}—X—R^{16a}, —C≡N, —NR₁₁ₐR^{12a}—C≡CH and —CH═CH₂; R^{9a} is selected from H and hydrocarbyl, R^{10a} is selected from H and hydrocarbyl; R^{11a} and R^{12a} are each independently selected from H and hydrocarbyl, q is an integer, X is O or S, R^{16a} is selected from H and hydrocarbyl and R^{17a} is selected from H and hydrocarbyl.

In some preferred aspects R³ is a group of the formula —(R^{7a})ₙR^{8a}, wherein n is 0 or 1, R^{7a} (CH₂)_{q}—X—R^{16a}, —C≡N, —NR^{11a}R^{12a}—C≡and —CH≡CH₂; R^{9a} is selected from H and hydrocarbyl, R^{10a} is selected from H and hydrocarbyl; R^{11a} and R^{12a} are each independently selected from H and hydrocarbyl; R^{13a} is selected from H and hydrocarbyl, q is an integer, X is O or S, R^{16a} is selected from H and hydrocarbyl and R^{17a} is selected from H and hydrocarbyl.

p may be any integer. p may be from 0 to 20. p may be from 0 to 10: Typically p is from 0 to 5. In one aspect p is 0, 1 or 2.

q may be any integer. q may be from 0 to 20. q may be from 0 to 10. Typically q is from 0 to 5. In one aspect q is 0, 1 or 2.

R^{8a} is selected from —SO₂—R^{9a}, —C(O)OR^{17a}, —OR^{10a}, (CH₂)_{q}—X—R^{16a}, —C≡N, —NR^{11a}R^{12a}—C≡CH and —CH═CH₂. In one preferred aspect R^{8a} is —SO₂—R^{9a}. In one preferred aspect R^{8a} is —SO₂—R^{9a} wherein R^{9a} is hydrocarbyl. Preferably in this aspect, R^{7a} is —O—, n is 1 and p is 0 such that R³ is —O—SO₂R^{9a}.

R^{9a} is selected from H and hydrocarbyl. In one aspect R^{9a} is hydrocarbyl. In one preferred embodiment of the present invention R^{9a} is selected from one of H, C₁-C₂₀ hydrocarbyl, C₁-C₁₀ hydrocarbyl, C₁-C₅ hydrocarbyl, C₁-C₃ hydrocarbyl, hydrocarbon groups, C₁-C₂₀ hydrocarbon, C₁-C₁₀ hydrocarbon, C₁-C₅ hydrocarbon, C₁-C₃ hydrocarbon, alkyl groups, C₁-C₂₀ alkyl, C₁-C₁₀ alkyl, C₁-C₅ alkyl, and C₁-C₃ alkyl.

In one aspect R^{9a} is selected from H and C₁₋₁₀ alkyl. In one aspect R^{9a} is C₁₋₁₀ alkyl. In one aspect R^{9a} is selected from H and C₁₋₅ alkyl. In one aspect R^{9a} is C₁₋₅ alkyl. In one aspect R^{9a} is selected from H and C₁₋₃ alkyl. In one aspect R^{9a} is C₁₋₃ alkyl. Preferably R^{9a} is —CH₂CH₃.

In one aspect R^{9a} is a substituted or unsubstituted amine. When substituted, the N-substituted compounds of this invention may contain one or two N-alkyl, N-alkenyl, N-cycloalkyl, N-acyl, or N-aryl substituents, preferably containing or each containing a maximum of 10 carbon atoms. In a preferred aspect, R^{9a} is an unsubstituted amine, i.e. R^{9a} is NH₂.

R^{10a} is selected from H and hydrocarbyl. In one preferred embodiment of the present invention R^{10a} is selected from one of H, C₁-C₂₀ hydrocarbyl, C₁-C₁₀ hydrocarbyl, C₁-C₅ hydrocarbyl, C₁-C₃ hydrocarbyl, hydrocarbon groups, C₁-C₂₀ hydrocarbon, C₁-C₁₀ hydrocarbon, C₁-C₅ hydrocarbon, C₁-C₃ hydrocarbon, alkyl groups, C₁-C₂₀ alkyl, C₁-C₁₀ alkyl, C₁-C₅ alkyl, and C₁-C₃ alkyl.

In one aspect R^{10a} is selected from H and C₁₋₁₀ alkyl. In one aspect R^{10a} is selected from H and C₁₋₅ alkyl. In one aspect R^{10a} is selected from H and C₁₋₃ alkyl. In one aspect R^{10a} is C₁₋₃ alkyl. Preferably R^{10a} is —H or —CH₃.

As previously mentioned, R^{11a} and R^{12a} of NR^{11a}R^{12a} are each independently selected from H and hydrocarbyl. When substituted, the N-substituted compounds of this invention may contain one or two N-alkyl, N-alkenyl, N-cycloalkyl, N-acyl, or N-aryl substituents, preferably containing or each containing a maximum of 10 carbon atoms. When R^{11a} and/or R^{12a} is alkyl, the preferred values are those where R^{11a} and R^{12a} are each independently selected from lower alkyl groups containing from 1 to 5 carbon atoms, that is to say methyl, ethyl, propyl etc. Preferably R^{11a} and R^{12a} are both methyl. When R^{11a} and/or R^{12a} is aryl, typical values are phenyl and tolyl (-PhCH₃; o-, m- or p-). Where R^{11a} and R^{12a} represent cycloalkyl, typical values are cyclopropyl, cyclopentyl, cyclohexyl etc. When joined together R^{11a} and R^{12a} typically represent an alkylene group providing a chain of 4 to 6 carbon atoms, optionally interrupted by one or more hetero atoms or groups, e.g. —O— or —NH— to provide a 5-, 6- or 7-membered heterocycle, e.g. morpholino, pyrrolidino or piperidino.

In one aspect R^{11a} and R^{12a} are each independently hydrocarbyl. In one preferred embodiment of the present invention R^{11a} and R^{12a} are each independently selected from one of H, C₁-C₂₀ hydrocarbyl, C₁-C₁₀ hydrocarbyl, C₁-C₅ hydrocarbyl, C₁-C₃ hydrocarbyl, hydrocarbon groups, C₁-C₂₀ hydrocarbon, C₁-C₁₀ hydrocarbon, C₁-C₅ hydrocarbon, C₁-C₃ hydrocarbon, alkyl groups, C₁-C₂₀ alkyl, C-C₁₀ alkyl, C₁-C₅ alkyl, and C₁-C₃ alkyl

In one aspect R^{11a} and R^{12a} are each independently selected from H and C₁₋₁₀ alkyl. In one aspect R^{11a} and R^{12a} are each independently C₁₋₁₀ alkyl. In one aspect R^{11a} and R^{12a} are each independently selected from H and C₁₋₅ alkyl. In one aspect R^{11a} and R^{12a} are each independently C₁₋₅ alkyl. In one aspect R^{11a} and R^{12a} are each independently selected from H and C₁₋₃ alkyl. In one aspect R^{11a} and R^{12a} are each independently C₁₋₃ alkyl. Preferably R^{1la} and R^{12a} are independently selected from —H and —CH₃.

R^{13a} is selected from H and hydrocarbyl. In one preferred embodiment of the present invention R^{13a} is selected from one of H, C₁-C₂₀ hydrocarbyl, C₁-C₁₀ hydrocarbyl, C₁-C₅ hydrocarbyl, C₁-C₃ hydrocarbyl, hydrocarbon groups, C₁-C₂₀ hydrocarbon, C₁-C₁₀ hydrocarbon, C₁-C₅ hydrocarbon, C₁-C₃ hydrocarbon, alkyl groups, C₁-C₂₀ alkyl, C₁-C₁₀ alkyl, C₁-C₅ alkyl, and C₁-C₃ alkyl.

In one aspect R^{13a} is selected from H and C₁₋₁₀ alkyl. In one aspect R^{13a} is selected from H and C₁₋₅ alkyl. In one aspect R^{13a} is selected from H and C₁₋₃ alkyl. In one aspect R^{13a} is C₁₋₃ alkyl. Preferably R^{13a} is —H.

R^{14a} and R^{15a} are each independently selected from H and hydrocarbyl. In one aspect R^{14a} and R^{15a} are each independently hydrocarbyl. In one preferred embodiment of the present invention R^{14a} and R^{15a} are each independently selected from one of H, C₁-C₂₀ hydrocarbyl, C₁-C₁₀ hydrocarbyl, C₁-C₅ hydrocarbyl, C₁-C₃ hydrocarbyl, hydrocarbon, groups, C₁-C₂₀ hydrocarbon, C₁-C₁₀ hydrocarbon, C₁-C₅ hydrocarbon, C₁-C₃ hydrocarbon, alkyl groups, C₁-C₂₀ alkyl, C₁-C₁₀ alkyl, C₁-C₅ alkyl, and C₁-C₃ alkyl.

In one aspect R^{14a} and R^{15a} are each independently selected from H and C₁₋₁₀ alkyl. In one aspect R^{14a} and R^{15a} are each independently C₁₋₁₀ alkyl. In one aspect R^{14a} and R^{15a} are each independently selected from H and C₁₋₅ alkyl. In one aspect R^{14a} and R^{15a} are each independently C₁₋₅ alkyl. In one aspect R^{14a} and R^{15a} are each independently selected from H and C₁₋₃ alkyl. In one aspect R^{14a} and R^{15a} are each independently C₁₋₃ alkyl.

Preferably R^{14a} and R^{15a} are independently selected from —H and —CH₃.

X is selected from O or S. In one aspect X is S. In one aspect X is O.

R^{18a} is selected from H and hydrocarbyl. In one preferred embodiment of the present invention R^{16a} is selected from one of H, C₁-C₂₀ hydrocarbyl, C₁-C₁₀ hydrocarbyl, C₁-C₅ hydrocarbyl, C₁-C₃ hydrocarbyl, hydrocarbon groups, C₁-C₂₀ hydrocarbon, C₁-C₁₀ hydrocarbon, C₁-C₅ hydrocarbon, C₁-C₃ hydrocarbon, alkyl groups, C₁-C₂₀ alkyl, C₁C₁₀ alkyl, C₁-C₅ alkyl, and C₁-C₃ alkyl.

In one aspect R^{16a} is selected from H and C₁₋₁₀ alkyl. In one aspect R^{16a} is selected from H and C₁₋₅ alkyl. In one aspect R^{16a} is selected from H and C₁₋₃ alkyl. In one aspect R^{16a} is C₁₋₃ alkyl. Preferably R^{16a} is —H.

In one highly preferred aspect R^{3a} is a group selected from ═CHC(O)OEt, —CH₂C(O)OEt, ═CHCH₂OH, —CH₂CH₂OH, —CH₂C≡N, ═CHC≡N, —NHCH₂CH₂N(Me)₂, —OCH₂CH₂—OMe.

In one aspect R^{3a} may be selected from the D substitutions shown below wherein each Q is independently selected from O, S, NH and CH₂ and y is an integer from 3 to 8, preferably 5, 6, 7 or 8.

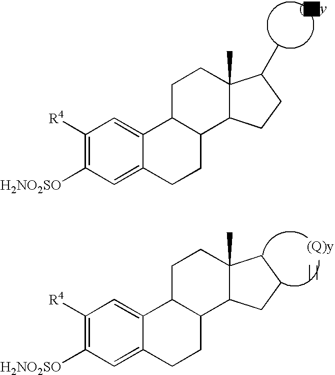

R⁴

As previously mentioned, the A ring of the steroidal ring system is optionally substituted with a group R⁴, wherein R⁴ is preferably selected from a hydrocarbyl group or an oxyhydrocarbyl group.

In one preferred embodiment of the present invention, the R⁴ is a oxyhydrocarbon group.

Here the term “oxyhydrocarbon” means, or R⁴ is, any one of an alkoxy group, an oxyalkenyl group, an oxyalkynyl group, which groups may be linear, branched or cyclic, or an oxyaryl group. The term oxyhydrocarbon also includes those groups but wherein they have been optionally substituted. If the oxyhydrocarbon is a branched structure having substituent(s) thereon, then the substitution may be on either the hydrocarbon backbone or on the branch; alternatively the substitutions may be on the hydrocarbon backbone and on the branch.

Preferably the oxyhydrocarbyl group R⁴ is an alkoxy group. Preferably the oxyhydrocarbyl group R⁴ is of the formula C₁₋₆O (such as a C₁₋₃O). Preferably the oxyhydrocarbyl group R⁴ is of the formula —O(CH₂)₁₋₁₀CH₃, —O(CH₂)₁₋₅CH₃, —O(CH₂)₁₋₂CH₃. In a highly preferred aspect R⁴ is methoxy.

Preferably the oxyhydrocarbyl group R⁴ is an ether group. Preferably the oxyhydrocarbyl group R⁴ is of the formula C₁₋₆OC₁₋₆ (such as a C₁₋₃OC₁₋₃). Preferably the oxyhydrocarbyl group R⁴ is of the formula —(CH₂)₁₋₁₀O(CH₂)₁₋₁₀CH₃, —(CH₂)₁₋₅O(CH₂₁₋₅CH₃, —(CH₂)₁₋₂O(CH₂)₁₋₂CH₃. In a highly preferred aspect R⁴ is —CH₂OCH₃.

In one preferred embodiment of the present invention, R⁴ is a hydrocarbon group. Preferably R⁴ is an alkyl group. Preferably the alkyl group is a C₁₋₆ alkyl group (such as a C₁₋₃ alkyl group). Preferably the hydrocarbyl group R⁴ is of the formula —(CH₂)₁₋₁₀CH₃, —(CH₂)₁₋₅CH₃, —(CH₂)₁₋₂CH₃. In a highly preferred aspect R⁴ is ethyl.

In one preferred embodiment of the present invention R⁴ is selected from one of C₁-C₁₀ hydrocarbyl, C₁-C₅ hydrocarbyl, C₁-C₃ hydrocarbyl, hydrocarbon groups, C₁-C₁₀ hydrocarbon, C₁-C₅ hydrocarbon, C₁-C₃ hydrocarbon, alkyl groups, C₁-C₁₀ alkyl, C₁-C₅ alkyl, and C₁-C₃ alkyl.

In one preferred embodiment of the present invention, the R⁴ is a hydrocarbylsulphanyl group.

The term “hydrocarbylsulphanyl” means a group that comprises at least hydrocarbyl group (as herein defined) and sulphur. That sulphur group may be optionally oxidised. Preferably the hydrocarbylsulphanyl is of the formula —S-hydrocarbyl wherein the hydrocarbyl is as described herein.

The term “hydrocarbylsulphanyl group” as used herein with respect to R⁴ means a group comprising at least C, H and S and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the hydrocarbylsulphanyl group comprises more than one C then those carbons need not necessarily be linked to each other. For example, at least two of the carbons may be linked via a suitable element or group. Thus, the hydrocarbylsulphanyl group may contain further hetero atoms. Suitable hetero atoms will be apparent to those skilled in the art and include, for instance, nitrogen.

In one preferred embodiment of the present invention, the R⁴ is a hydrocarbonsulphanyl group. The term “hydrocarbonsulphanyl group” as used herein with respect to R⁴ means a group consisting of C, H and S. Preferably the hydrocarbonsulphanyl is of the formula —S— hydrocarbon wherein the hydrocarbon is as described herein.

Preferably the hydrocarbonsulphanyl group R⁴ is of the formula C₁₋₆S (such as a C₁₋₃S). Preferably the oxyhydrocarbyl group R⁴ is of the formula —S(CH₂)₁₋₁₀CH₃, —S(CH₂)₁₋₅CH₃, —S(CH₂)₁₋₂CH₃. In a highly preferred aspect R⁴ is —S—Me.

As previously mentioned, R⁴ is at position 2 or 4 of the A ring. Thus the compound may have the formula

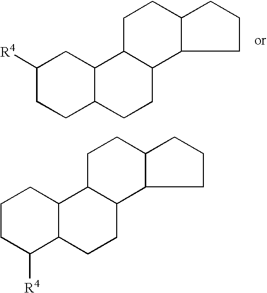

wherein R¹ and R² are as specified herein, such as

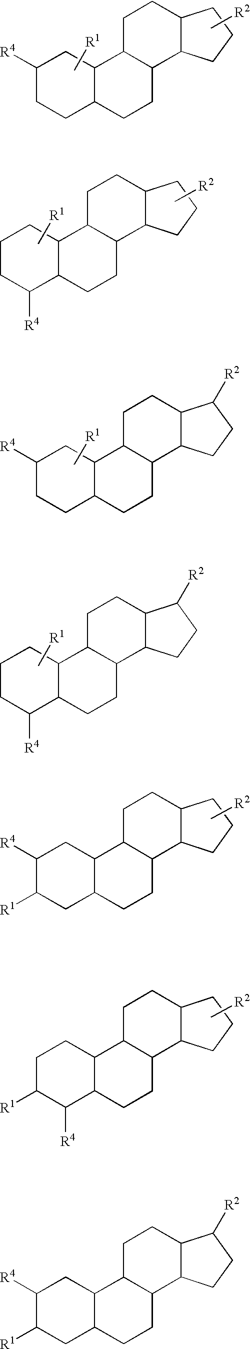

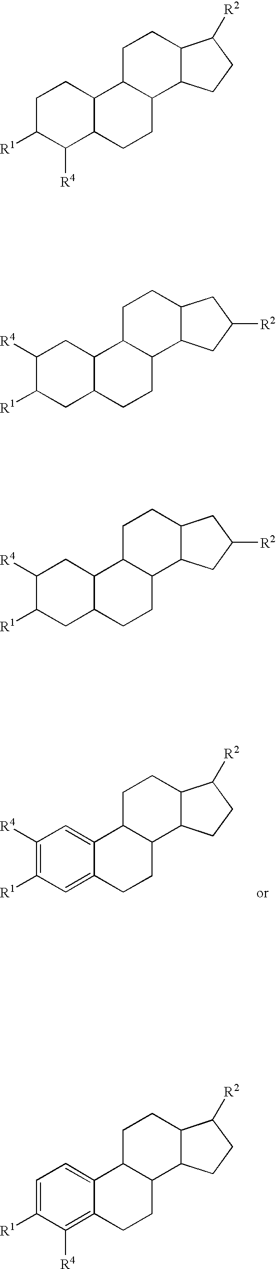

Preferably R⁴ is at position 2 of the A ring.

In a further preferred aspect when the A ring is substituted with R¹ and R⁴, R⁴ is ortho with respect to R¹.

It will be appreciated by one skilled in the art that the proviso that R⁴ is at position 2 or 4 of the A ring, allows for R⁴ being at position 2 and 4 of the A ring, wherein each R⁴ is independently selected from the possibilities recited herein.

In a highly preferred aspect the compound is of Formula Va

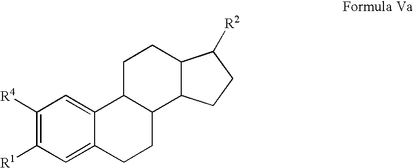

wherein R¹ is selected from —OH and a sulphamate group

wherein R² is selected from —OH, a sulphamate group,

wherein the or each sulphamate group is of the formula

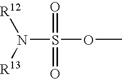

wherein R¹² and R¹³ are independently selected from H and alkyl.

and wherein R⁴ is an alkoxy group.

In a highly preferred aspect the compound is selected from

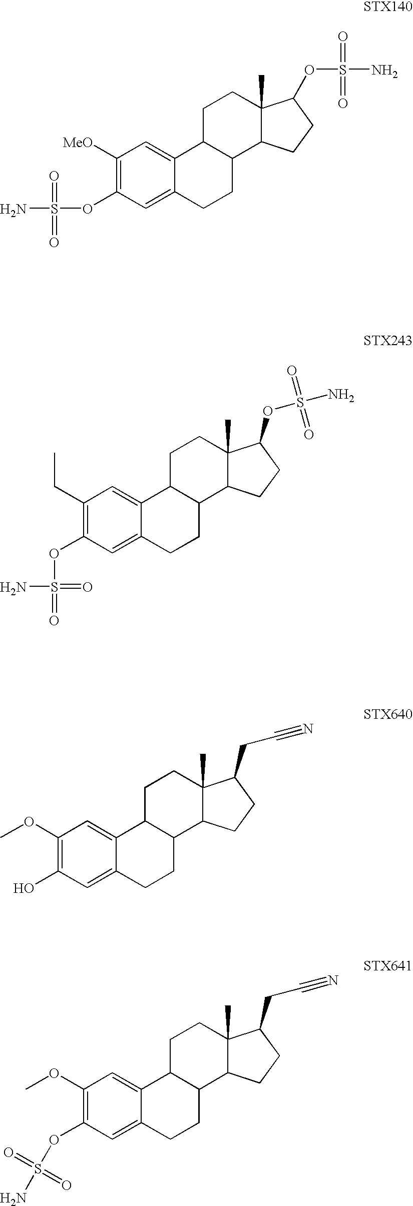

In a highly preferred aspect the compound is

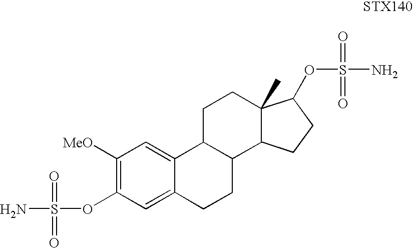

Distinct from Docetaxel, 2-Methoxyestradiol-3,17-O,O-bis-sulfamate (STX140) is a microtubule disruptor (Raobaikady 2005) which is orally bioavailable (Ireson 2004), not a substrate for p-glycoprotein (Suzuki, 2003) and can be dosed daily in vivo (Foster, submitted; Ireson 2004). The microtubule disruption leads to cell cycle arrest and subsequent apoptosis in both tumour and endothelial cells and inhibits in vitro angiogenesis (Newman et al., 2004) and in vivo angiogenesis (Foster, submitted; and Chander submitted).

STX140, was developed from the original steroid sulfatase (STS) inhibitor EMATE in order to overcome estrogenicity problems (Purohit et al, 1995-Biochemistry). The new class of A-ring modified anti-cancer compounds not only inhibited STS but were potent inhibitors of cell proliferation in vitro and in NMU induced tumours in vivo (Purohit et al, 2000, Int J Cancer).

The structurally related compound 2-MeOE2 is a well studied compound with known anti-cancer properties, and can inhibit cell proliferation and angiogenesis via a mechanism independent of hormone receptors (Fotsis et al., 1994). However, the clinical success of 2-MeOE2 has been limited as it has very poor oral bioavailability and is rapidly metabolised. In contrast to 2-MeOE2, STX140 avoids metabolism by 17βHSB due to its sulfate moiety at the C-17 position (Newman et al., 2006). STX140 has previously been shown to inhibit the proliferation of prostate LNCaP (androgen, AR+ve) cells (Day et al., 2003) and breast ER+ve MCF-7 cells (Suzuki et al., 2003b). STX140 inhibits doxorubicin and mitoxantrone resistant MCF-7 breast cancer proliferation (Suzuki et al., 2003a). The inhibition of in vivo angiogenesis by STX140 will increase hypoxia and make the tumour more reliant on glycolysis, thus sensitising the tumour to 2DG.

Composition

As described above according to one aspect of the present invention, there is provided a pharmaceutical composition comprising a composition as defined herein, and a pharmaceutically acceptable carrier, diluent, excipient or adjuvant.

In accordance with the present invention the composition of the present invention may comprise more than one biological response modifier.

The term biological response modifier (“BRM”) includes cytokines, immune modulators, growth factors, haematopoiesis regulating factors, colony stimulating factors, chemotactic, haemolytic and thrombolytic factors, cell surface receptors, ligands, leukocyte adhesion molecules, monoclonal antibodies, preventative and therapeutic vaccines, hormones, extracellular matrix components, fibronectin, etc.

BRMs may play a role in modulating the immune and inflammatory response in disorders. Examples of BRMs include: Tumour Necrosis Factor (TNF), granulocyte colony stimulating factor, erythropoietin, insulin-like growth factor (IGF), epidermal growth factor (EGF), transforming growth factor (TGF), platelet-derived growth factor (PDGF), interferons (IFNs), interleukins, tissue plasminogen activators, P-, E- or L-Selectins, ICAM-1, VCAM, Selectins, addressins etc.

Preferably, the biological response modifier is a cytokine.

A cytokine is a molecule—often a soluble protein—that allows immune cells to communicate with each other. These molecules exert their biological functions through specific receptors expressed on the surface of target cells. Binding of the receptors triggers the release of a cascade of biochemical signals which profoundly affect the behaviour of the cell bearing the receptor (Poole, S 1995 TibTech 13: 81-82). Many cytokines and their receptors have been identified at the molecular level (Paul and Sedar 1994, Cell 76: 241-251) and make suitable molecules of therapeutic value as well as therapeutic targets in their own right.

More details on cytokines can be found in Molecular Biology and Biotechnology (Pub. VCH, Ed. Meyers, 1995, pages 202, 203, 394, 390, 475, 790).

Examples of cytokines include: interleukins (IL)—such as IL-1, IL-2, IL-3, IL-4, IL-5, IL-6, IL-7, IL-8; IL-9, IL-10, IL-11, IL-12, IL-19; Tumour Necrosis Factor (TNF)—such as TNF-α; Interferon alpha, beta and gamma; TGF-β.

For the present invention, preferably the cytokine is tumour necrosis factor (TNF).

More preferably the cytokine is TNF-a.

TNF is a cytokine produced by macrophages and lymphocytes which mediates inflammatory and immunopathological responses. TNF has been implicated in the progression of diseases which include but are not limited to immunomodulation disorder, infection, cell proliferation, angiogenesis (neovascularisation), tumour metastasis, apoptosis, sepsis, and endotoxaemia.

The necrotising action of TNF in vivo mainly relates to capillary injury. TNF causes necrosis not only in tumour tissue but also in granulation tissue. It causes morphological changes in growth inhibition of and cytoxicity against cultured vascular endothelial cells (Haranka et al 1987 Ciba Found Symp 131: 140-153).

For the preferred aspect of the present invention, the TNF may be any type of TNF—such as TNF-α, TNF-β, including derivatives or mixtures thereof.

Teachings on TNF may be found in the art—such as WO-A-98/08870 and WO-A-98/13348.

The TNF can be prepared chemically or it can be extracted from sources. Preferably, the TNF is prepared by use of recombinant DNA techniques.

With this aspect of the present invention the compositions of the present invention are more potent in vivo than the compounds alone or TNF alone. Moreover, in some aspects the combination of compounds and TNF is more potent than one would expect from the potency of the compound alone i.e. this is a synergistic relationship between them.

In addition, the present invention contemplates the composition of the present invention further comprising an inducer of the biological response modifier—such as in vivo inducer of the biological response modifier.

In accordance with the present invention, the components of the composition can be added in admixture, simultaneously or sequentially. Furthermore, in accordance with the present invention it may be possible to form at least a part of the composition in situ (such as in vivo) by inducing the expression of—or increasing the expression of—one of the components. For example, it may be possible to induce the expression of—or increase the expression of—the biological response modifier, such as TNF. By way of example, it may be possible to induce the expression of—or increase the expression of—TNF by adding bacterial lipopolysaccharide (LPS) and muramyl dipeptide (MDP). In this regard, bacterial LPS and MDP in combination can stimulate TNF production from murine spleen cells in vitro and tumour regression in vivo (Fuks et al Biull Eksp Biol Med 1987 104: 497-499).

In the method of treatment, the subject is preferably a mammal, more preferably a human. For some applications, preferably the human is a woman.

The present invention also covers novel intermediates that are useful to prepare the compounds of the present invention. For example, the present invention covers novel alcohol precursors for the compounds. By way of further example, the present invention covers bis protected precursors for the compounds. Examples of each of these precursors are presented herein. The present invention also encompasses a process comprising each or both of those precursors for the synthesis of the compounds of the present invention.

Therapy

The compounds of the present invention may be used as therapeutic agents—i.e. In therapy applications.

The term “therapy” includes curative effects, alleviation effects, and prophylactic effects.

The therapy may be on humans or animals, preferably female animals.

Pharmaceutical Compositions

In one aspect, the present invention provides a pharmaceutical composition, which comprises a composition according to the present invention and optionally a pharmaceutically acceptable carrier, diluent or excipient (including combinations thereof).

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilisers, dyes and even flavouring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be delivered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be delivered by both routes.

Where the agent is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

Combination Pharmaceutical

The compound of the present invention may be used in combination with one or more other active agents, such as one or more other pharmaceutically active agents.

By way of example, the compounds of the present invention may be used in combination with other STS inhibitors and/or other inhibitors such as an aromatase inhibitor (such as for example, 4-hydroxyandrostenedione (4-OHA)) and/or steroids—such as the naturally occurring neurosteroids dehydroepiandrosterone sulfate (DHEAS) and pregnenolone sulfate (PS) and/or other structurally similar organic compounds. Examples of other STS inhibitors may be found in the above references. By way of example, STS inhibitors for use in the present invention include EMATE, and either or both of the 2-ethyl and 2-methoxy 17-deoxy compounds that are analogous to compound 5 presented herein.

In addition, or in the alternative, the compound of the present invention may be used in combination with a biological response modifier.

The term biological response modifier (“BRM”) includes cytokines, immune modulators, growth factors, haematopoiesis regulating factors, colony stimulating factors, chemotactic, haemolytic and thrombolytic factors, cell surface receptors, ligands, leukocyte adhesion molecules, monoclonal antibodies, preventative and therapeutic vaccines, hormones, extracellular matrix components, fibronectin, etc. For some applications, preferably, the biological response modifier is a cytokine. Examples of cytokines include: interleukins (IL)—such as IL-1, IL-2, IL-3, IL-4, IL-5, IL-6, IL-7, IL-8, IL-9, IL-10, IL-11, IL-12, IL-19; Tumour Necrosis Factor (TNF)—such as TNF-α; Interferon alpha, beta and gamma; TGF-β. For some applications, preferably the cytokine is tumour necrosis factor (TNF). For some applications, the TNF may be any type of TNF—such as TNF-α, TNF-β, including derivatives or mixtures thereof. More preferably the cytokine is TNF-α. Teachings on TNF may be found in the art—such as WO-A-98/08870 and WO-A-98/13348.

Administration

Typically, a physician will determine the actual dosage which will be most suitable for an individual subject and it will vary with the age, weight and response of the particular patient. The dosages below are exemplary of the average case. There can, of course, be individual instances where higher or lower dosage ranges are merited.

The compositions of the present invention may be administered by direct injection. The composition may be formulated for parenteral, mucosal, intramuscular, intravenous, subcutaneous, intraocular or transdermal administration. Depending upon the need, the agent may be administered at a dose of from 0.01 to 30 mg/kg body weight, such as from 0.1 to 10 mg/kg, more preferably from 0.1 to 1 mg/kg body weight.

By way of further example, the agents of the present invention may be administered in accordance with a regimen of 1 to 4 times per day, preferably once or twice per day. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Aside from the typical modes of delivery—indicated above—the term “administered” also includes delivery by techniques such as lipid mediated transfection, liposomes, immunoliposomes, lipofectin, cationic facial amphiphiles (CFAs) and combinations thereof. The routes for such delivery mechanisms include but are not limited to mucosal, nasal, oral, parenteral, gastrointestinal, topical, or sublingual routes.

The term “administered” includes but is not limited to delivery by a mucosal route, for example, as a nasal spray or aerosol for inhalation or as an ingestable solution; a parenteral route where delivery is by an injectable form, such as, for example, an intravenous, intramuscular or subcutaneous route.

Thus, for pharmaceutical administration, the STS inhibitors of the present invention can be formulated in any suitable manner utilising conventional pharmaceutical formulating techniques and pharmaceutical carriers, adjuvants, excipients, diluents etc. and usually for parenteral administration. Approximate effective dose rates may be in the range from 1 to 1000 mg/day, such as from 10 to 900 mg/day or even from 100 to 800 mg/day depending on the individual activities of the compounds in question and for a patient of average (70Kg) bodyweight. More usual dosage rates for the preferred and more active compounds will be in the range 200 to 800 mg/day, more preferably, 200 to 500 mg/day, most preferably from 200 to 250 mg/day. They may be given in single dose regimes, split dose regimes and/or in multiple dose regimes lasting over several days. For oral administration they may be formulated in tablets, capsules, solution or suspension containing from 100 to 500 mg of compound per unit dose. Alternatively and preferably the compounds will be formulated for parenteral administration in a suitable parenterally administrable carrier and providing single daily dosage rates in the range 200 to 800 mg, preferably 200 to 500, more preferably 200 to 250 mg. Such effective daily doses will, however, vary depending on inherent activity of the active ingredient and on the bodyweight of the patient, such variations being within the skill and judgement of the physician.

Cell Cycling

The compounds of the present invention may be useful in the method of treatment of a cell cycling disorder.

As discussed in “Molecular Cell Biology” 3rd Ed. Lodish at al. pages 177-181 different eukaryotic cells can grow and divide at quite different rates. Yeast cells, for example, can divide every 120 min., and the first divisions of fertilised eggs in the embryonic cells of sea urchins and insects take only 1530 min. because one large pre-existing cell is subdivided. However, most growing plant and animal cells take 10-20 hours to double in number, and some duplicate at a much slower rate. Many cells in adults, such as nerve cells and striated muscle cells, do not divide at all; others, like the fibroblasts that assist in healing wounds, grow on demand but are otherwise quiescent.

Still, every eukaryotic cell that divides must be ready to donate equal genetic material to two daughter cells. DNA synthesis in eukaryotes does not occur throughout the cell division cycle but is restricted to a part of it before cell division.

The relationship between eukaryotic DNA synthesis and cell division has been thoroughly analysed in cultures of mammalian cells that were all capable of growth and division. In contrast to bacteria, it was found, eukaryotic cells spend only a part of their time in DNA synthesis, and it is completed hours before cell division (mitosis). Thus a gap of time occurs after DNA synthesis and before cell division; another gap was found to occur after division and before the next round of DNA synthesis. This analysis led to the conclusion that the eukaryotic cell cycle consists of an M (mitotic) phase, a G₁ phase (the first gap), the S (DNA synthesis) phase, a G₂ phase (the second gap), and back to M. The phases between mitoses (G₁, S, and G₂) are known collectively as the interphase.

Many nondividing cells in tissues (for example, all quiescent fibroblasts) suspend the cycle after mitosis and just prior to DNA synthesis; such “resting” cells are said to have exited from the cell cycle and to be in the G₀ state.

It is possible to identify cells when they are in one of the three interphase stages of the cell cycle, by using a fluorescence-activated cell sorter (FACS) to measure their relative DNA content: a cell that is in G₁ (before DNA synthesis) has a defined amount x of DNA; during S (DNA replication), it has between x and 2x; and when in G₂ (or M), it has 2x of DNA.

The stages of mitosis and cytokinesis in an animal cell are as follows

(a) Interphase. The G₂ stage of interphase immediately precedes the beginning of mitosis. Chromosomal DNA has been replicated and bound to protein during the S phase, but chromosomes are not yet seen as distinct structures. The nucleolus is the only nuclear substructure that is visible under light microscope. In a diploid cell before DNA replication there are two morphologic chromosomes of each type, and the cell is said to be 2n. In G₂, after DNA replication, the cell is 4n. There are four copies of each chromosomal DNA. Since the sister chromosomes have not yet separated from each other, they are called sister chromatids.

b) Early prophase. Centrioles, each with a newly formed daughter centriole, begin moving toward opposite poles of the cell; the chromosomes can be seen as long threads. The nuclear membrane begins to disaggregate into small vesicles.

(c) Middle and late prophase. Chromosome condensation is completed; each visible chromosome structure is composed of two chromatids held together at their centromeres. Each chromatid contains one of the two newly replicated daughter DNA molecules. The microtubular spindle begins to radiate from the regions just adjacent to the centrioles, which are moving closer to their poles. Some spindle fibres reach from pole to pole; most go to chromatids and attach at kinetochores.

(d) Metaphase. The chromosomes move toward the equator of the cell, where they become aligned in the equatorial plane. The sister chromatids have not yet separated.

(e) Anaphase. The two sister chromatids separate into independent chromosomes. Each contains a centromere that is linked by a spindle fibre to one pole, to which it moves. Thus one copy of each chromosome is donated to each daughter cell. Simultaneously, the cell elongates, as do the pole-to-pole spindles. Cytokinesis begins as the cleavage furrow starts to form.

(f) Telophase. New membranes form around the daughter nuclei; the chromosomes uncoil and become less distinct, the nucleolus becomes visible again, and the nuclear membrane forms around each daughter nucleus. Cytokinesis is nearly complete, and the spindle disappears as the microtubules and other fibres depolymerise. Throughout mitosis the “daughter” centriole at each pole grows until it is full-length. At telophase the duplication of each of the original centrioles is completed, and new daughter centrioles will be generated during the next interphase.

(g) Interphase. _Upon the completion of cytokinesis, the cell enters the G₁ phase of the cell cycle and proceeds again around the cycle.

It will be appreciated that cell cycling is an extremely important cell process. Deviations from normal cell cycling can result in a number of medical disorders. Increased and/or unrestricted cell cycling may result in cancer. Reduced cell cycling may result in degenerative conditions. Use of the compound of the present invention may provide a means to treat such disorders and conditions.

Thus, the compound of the present invention may be suitable for use in the treatment of cell cycling disorders such as cancers, including hormone dependent and hormone independent cancers.

In addition, the compound of the present invention may be suitable for the treatment of cancers such as breast cancer, ovarian cancer, endometrial cancer, sarcomas, melanomas, prostate cancer, pancreatic cancer etc. and other solid tumours.

For some applications, cell cycling is inhibited and/or prevented and/or arrested, preferably wherein cell cycling is prevented and/or arrested. In one aspect cell cycling may be inhibited and/or prevented and/or arrested in the G₂/M phase. In one aspect cell cycling may be irreversibly prevented and/or inhibited and/or arrested, preferably wherein cell cycling is irreversibly prevented and/or arrested.

By the term “irreversibly prevented and/or inhibited and/or arrested” it is meant after application of a compound of the present invention, on removal of the compound the effects of the compound, namely prevention and/or inhibition and/or arrest of cell cycling, are still observable. More particularly by the term “irreversibly prevented and/or inhibited and/or arrested” it is meant that when assayed in accordance with the cell cycling assay protocol presented herein, cells treated with a compound of interest show less growth after Stage 2 of the protocol I than control cells. Details on this protocol are presented below.

Thus, the present invention provides compounds which: cause inhibition of growth of oestrogen receptor positive (ER+) and ER negative (ER−) breast cancer cells in vitro by preventing and/or inhibiting and/or arresting cell cycling; and/or cause regression of nitroso-methyl urea (NMU)-induced mammary tumours in intact animals (i.e. not ovariectomised), and/or prevent and/or inhibit and/or arrest cell cycling in cancer cells; and/or act in vivo by preventing and/or inhibiting and/or arresting cell cycling and/or act as a cell cycling agonist.

### Cell Cycling Assay

Procedure

Stage 1

MCF-7 breast cancer cells are seeded into multi-well culture plates at a density of 105 cells/well. Cells were allowed to attach and grown until about 30% confluent when they are treated as follows:

Control—no treatment

Compound of Interest (COI) 20 μM

Cells are grown for 6 days in growth medium containing the COI with changes of medium/COI every 3 days. At the end of this period cell numbers were counted using a Coulter cell counter.

Stage 2

After treatment of cells for a 6-day period with the COI cells are re-seeded at a density of 10⁴ cells/well. No further treatments are added. Cells are allowed to continue to grow for a further 6 days in the presence of growth medium. At the end of this period cell numbers are again counted.

Cancer

As indicated, the compounds of the present invention may be useful in the treatment of a cell cycling disorder. A particular cell cycling disorder is cancer.

Cancer remains a major cause of mortality in most Western countries. Cancer therapies developed so far have included blocking the action or synthesis of hormones to inhibit the growth of hormone-dependent tumours. However, more aggressive chemotherapy is currently employed for the treatment of hormone-independent tumours.

Hence, the development of a pharmaceutical for anti-cancer treatment of hormone dependent and/or hormone independent tumours, yet lacking some or all of the side-effects associated with chemotherapy, would represent a major therapeutic advance.

It is known that oestrogens undergo a number of hydroxylation and conjugation reactions after their synthesis. Until recently it was thought that such reactions were part of a metabolic process that ultimately rendered oestrogens water soluble and enhanced their elimination from the body. It is now evident that some hydroxy metabolites (e.g. 2-hydroxy and 16alpha-hydroxy) and conjugates (e.g. oestrone sulphate, EIS) are important in determining some of the complex actions that oestrogens have in the body.

Workers have investigated the formation of 2- and 16-hydroxylated oestrogens in relation to conditions that alter the risk of breast cancer. There is now evidence that factors which increase 2-hydroxylase activity are associated with a reduced cancer risk, while those increasing 16alpha-hydroxylation may enhance the risk of breast cancer. Further interest in the biological role of estrogen metabolites has been stimulated by the growing body of evidence that 2-methoxyoestradiol is an endogenous metabolite with anti-mitotic properties. 2-MeOE2 is formed from 2-hydroxy estradiol (2—OHE2) by catechol estrogen methyl transferase, an enzyme that is widely distributed throughout the body.

Workers have shown that in vivo 2-MeOE2 inhibits the growth of tumours arising from the subcutaneous injection of Meth A sarcoma, B16 melanoma or MDA-MB-435 estrogen receptor negative (ER−) breast cancer cells. It also inhibits endothelial cell proliferation and migration, and in vitro angiogenesis. It was suggested that the ability of 2-MeOE2 to inhibit tumour growth in vivo may be due to its ability to inhibit tumour-induced angiogenesis rather than direct inhibition of the proliferation of tumour cells.

The mechanism by which 2-MeOE2 exerts its potent anti-mitogenic and anti-angiogenic effects is still being elucidated. There is evidence that at high concentrations it can inhibit microtubule polymerisation and act as a weak inhibitor of colchicine binding to tubulin. Recently, however, at concentrations that block mitosis, tubulin filaments in cells were not found to be depolymerised but to have an identical morphology to that seen after taxol treatment. It is possible, therefore, that like taxol, a drug that is used for breast and ovarian breast cancer therapy, 2-MeOE2 acts by stabilising microtubule dynamics.

While the identification of 2-MeOE2 as a new therapy for cancer represents an important advance, the bioavailability of orally administered oestrogens is poor. Furthermore, they can undergo extensive metabolism during their first pass through the liver. As part of a research programme to develop a steroid sulphatase inhibitor for breast cancer therapy, oestrone-3-O-sulphamate (EMATE) was identified as a potent active site-directed inhibitor. Unexpectedly, EMATE proved to possess potent oestrogenic properties with its oral uterotrophic activity in rats being a 100-times higher than that of estradiol. Its enhanced oestrogenicity is thought to result from its absorption by red blood cells (rbcs) which protects it from inactivation during its passage through the liver and which act as a reservoir for its slow release for a prolonged period of time. A number of A-ring modified analogues were synthesised and tested, including 2-methoxyoestrone-3-O-sulphamate. While this compound was equipotent with EMATE as a steroid sulphatase inhibitor, it was devoid of oestrogenicity.

We believe that the compound of the present invention provides a means for the treatment of cancers and, especially, breast cancer.

In addition or in the alternative the compound of the present invention may be useful in the blocking the growth of cancers including leukaemias and solid tumours such as breast, endometrium, prostate, ovary and pancreatic tumours.

Therapy Concerning Oestrogen

We believe that some of the compounds of the present invention may be useful in the control of oestrogen levels in the body—in particular in females. Thus, some of the compounds may be useful as providing a means of fertility control—such as an oral contraceptive tablet, pill, solution or lozenge. Alternatively, the compound could be in the form of an implant or as a patch.

Thus, the compounds of the present invention may be useful in treating hormonal conditions associated with oestrogen.

In addition or in the alternative the compound of the present invention may be useful in treating hormonal conditions in addition to those associated with oestrogen. Hence, the compound of the present invention may also be capable of affecting hormonal activity and may also be capable of affecting an immune response.

Neurodegenerative Diseases

We believe that some of the compounds of the present invention may be useful in the treatment of neurodegenerative diseases, and similar conditions.

By way of example, it is believed that STS inhibitors may be useful in the enhancing the memory function of patients suffering from illnesses such as amnesia, head. injuries, Alzheimer's disease, epileptic dementia, presenile dementia, post traumatic dementia, senile dementia, vascular dementia and post-stroke dementia or individuals otherwise seeking memory enhancement.

TH1

We believe that some of the compounds of the present invention may be useful in TH1 implications.

By way of example, it is believed that the presence of STS inhibitors within the macrophage or other antigen presenting cells may lead to a decreased ability of sensitised T cells to mount a TH1 (high IL-2, IFNγ low IL-4) response. The normal regulatory influence of other steroids such as glucocorticoids would therefore predominate.

Inflamatory Conditions

We believe that some of the compounds of the present invention may be useful in treating inflammatory conditions—such as conditions associated with any one or more of: autoimmunity, including for example, rheumatoid arthritis, type I and II diabetes, systemic lupus erythematosus, multiple sclerosis, myasthenia gravis, thyroiditis, vasculitis, ulcerative colitis and Crohn's disease, skin disorders e.g. psoriasis and contact dermatitis; graft versus host disease; eczema; asthma and organ rejection following transplantation.

By way of example, it is believed that STS inhibitors may prevent the normal physiological effect of DHEA or related steroids on immune and/or inflammatory responses.

The compounds of the present invention may be useful in the manufacture of a medicament for revealing an endogenous glucocorticoid-like effect.

Other Therapies

It is also to be understood that the compound/composition of the present invention may have other important medical implications.

For example, the compound or composition of the present invention may be useful in the treatment of the disorders listed in WO-A-99/52890—viz:

In addition, or in the alternative, the compound or composition of the present invention may be useful in the treatment of the disorders listed in WO-A-98/05635. For ease of reference, part of that list is now provided: cancer, inflammation or inflammatory disease, dermatological disorders, fever, cardiovascular effects, haemorrhage, coagulation and acute phase response, cachexia, anorexia, acute infection, HIV infection, shock states, graft-versus-host reactions, autoimmune disease, reperfusion injury, meningitis, migraine and aspirin-dependent anti-thrombosis; tumour growth, invasion and spread, angiogenesis, metastases, malignant, ascites and malignant pleural effusion; cerebral ischaemia, ischaemic heart disease, osteoarthritis, rheumatoid arthritis, osteoporosis, asthma, multiple sclerosis, neurodegeneration, Alzheimer's disease, atherosclerosis, stroke, vasculitis, Crohn's disease and ulcerative colitis; periodontitis, gingivitis; psoriasis, atopic dermatitis, chronic ulcers, epidermolysis bullosa; corneal ulceration, retinopathy and surgical wound healing; rhinitis, allergic conjunctivitis, eczema, anaphylaxis; restenosis, congestive heart failure, endometriosis, atherosclerosis or endosclerosis.

In addition, or in the alternative, the compound or composition of the present invention may be useful in the treatment of disorders listed in WO-A-98/07859. For ease of reference, part of that list is now provided: cytokine and cell proliferation/differentiation activity; immunosuppressant or immunostimulant activity (e.g. for treating immune deficiency, including infection with human immune deficiency virus; regulation of lymphocyte growth; treating cancer and many autoimmune diseases, and to prevent transplant rejection or induce tumour immunity); regulation of haematopoiesis, e.g. treatment of myeloid or lymphoid diseases; promoting growth of bone, cartilage, tendon, ligament and nerve tissue, e.g. for healing wounds, treatment of burns, ulcers and periodontal disease and neurodegeneration; inhibition or activation of follicle-stimulating hormone (modulation of fertility); chemotactic/chemokinetic activity (e.g. for mobilising specific cell types to sites of injury or infection); haemostatic and thrombolytic activity (e.g. for treating haemophilia and stroke); antiinflammatory activity (for treating e.g. septic shock or Crohn's disease); as antimicrobials; modulators of e.g. metabolism or behaviour; as analgesics; treating specific deficiency disorders; in treatment of e.g. psoriasis, in human or veterinary medicine.

In addition, or in the alternative, the composition of the present invention may be useful in the treatment of disorders listed in WO-A-98/09985. For ease of reference, part of that list is now provided: macrophage inhibitory and/or T cell inhibitory activity and thus, anti- inflammatory activity; anti-immune activity, i.e. inhibitory effects against a cellular and/or humoral immune response, including a response not associated with inflammation; inhibit the ability of macrophages and T cells to adhere to extracellular matrix components and fibronectin, as well as up-regulated fas receptor expression in T cells; inhibit unwanted immune reaction and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery, bone marrow transplantation or other transplantation complications and/or side effects, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

In addition, or in the alternative, the compound or composition of the present invention may be useful in the treatment of the disorders listed selected from endometriosis, uterus fibromyoma, induction of mono-ovulation (in polycystic ovarian disease [PCOD] patients). induction of multiple follicullar development in (ART patients), preterm labor/cervical incompetency and recurrent abortion.

Compound Preparation

The compounds may be prepared by reacting an appropriate alcohol with a suitable chloride. By way of example, the sulphamate compounds of the present invention may be prepared by reacting an appropriate alcohol with a suitable sulfamoyl chloride, of the formula R⁴R⁵NSO₂Cl. Specific methods for preparing the compounds are provided in WO-A-02/16392, WO-A-2004/085459 and WO-A-2006/032885.

Typical conditions for carrying out the reaction are as follows.

Sodium hydride and a sulfamoyl chloride are added to a stirred solution of the alcohol in anhydrous dimethyl formamide at 0° C. Subsequently, the reaction is allowed to warm to room temperature whereupon stirring is continued for a further 24 hours. The reaction mixture is poured onto a cold saturated solution of sodium bicarbonate and the resulting aqueous phase is extracted with dichloromethane. The combined organic extracts are dried over anhydrous MgSO₄. Filtration followed by solvent evaporation in vacuo and co-evaporated with toluene affords a crude residue which is further purified by flash chromatography.

Preferably, the alcohol is derivatised, as appropriate, prior to reaction with the sulfamoyl chloride. Where necessary, functional groups in the alcohol may be protected in known manner and the protecting group or groups removed at the end of the reaction.

Preferably, the sulphamate compounds are prepared according to the teachings of Page et al (1990 Tetrahedron 46; 2059-2068).

### Examples

The present invention will now be described in further detail by way of example only with reference to the accompanying figures in which:—

FIG. 1 shows: Structural difference between glucose and 2-deoxy-D-glucose. Glucose and 2DG differ at the second carbon. (B) Chemical structure of STX140.

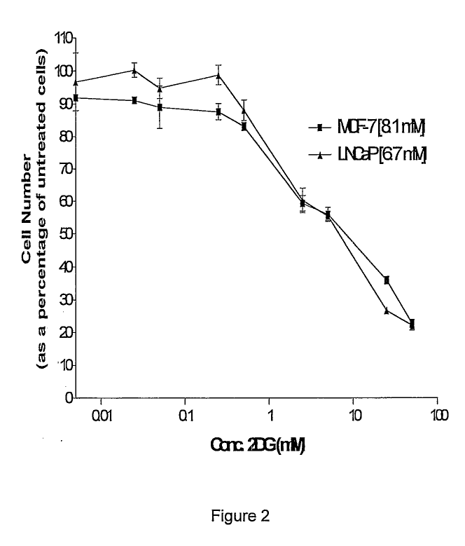

FIG. 2 shows: Effect of 2DG on the proliferation of LNCaP and MCF-7 cells. Cells were cultured in 96-well plates and treated with 2DG (0.005 mM-50 mM) for 5 days when their effects on proliferation were measured using a microtiter plate assay.

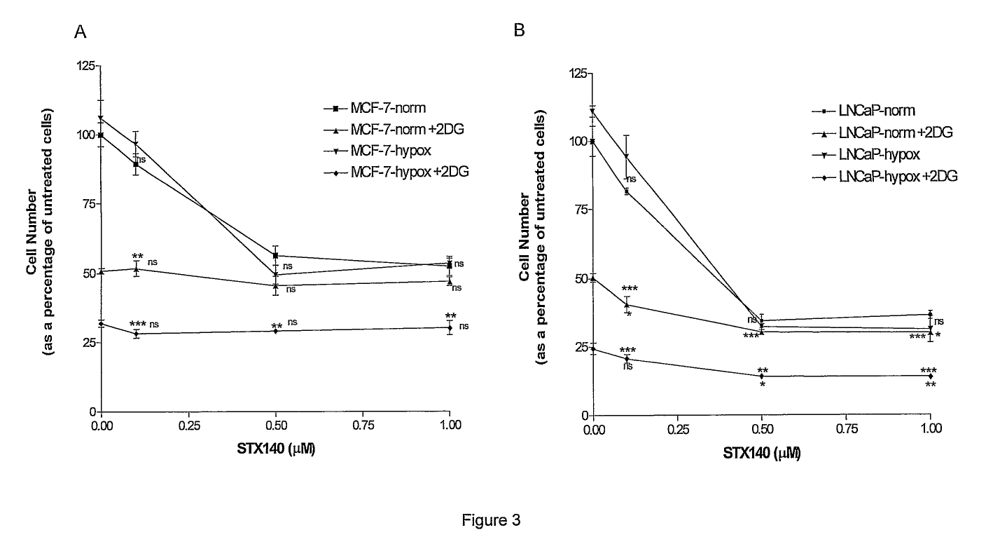

FIG. 3 shows: Effect of 2DG and STX140 on the proliferation of LNCaP (B) and MCF-7 (A) cells. Cells were cultured in 96-well plates, under normoxia or hypoxia, and treated with 2DG (8 mM) and STX140 (0.1 μM-1 μM) for 3 days when their effects on proliferation were measured using a microtiter plate assay. A two-tailed Student's t-test assuming unequal variance was carried out, comparing values to normoxic untreated controls (black) or to 2DG (8 mM) alone (grey). ns=not significant; *p<0.05; **p<0.01; ***p<0.001

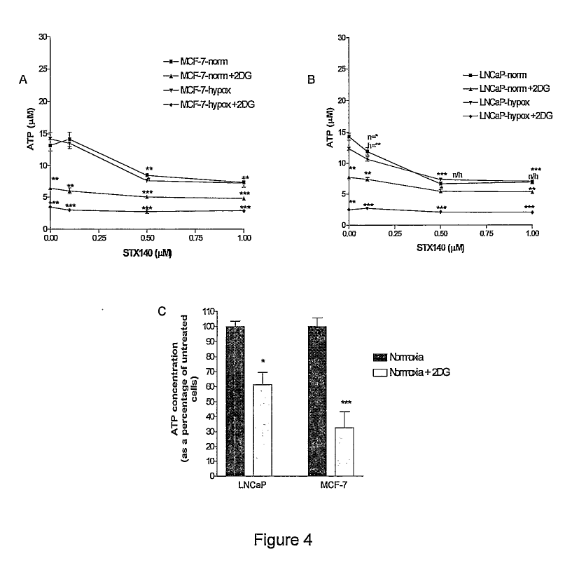

FIG. 4 shows: Effect of 2DG and STX140 on the ATP levels of LNCaP (FIG. 4B) and MCF-7 (FIG. 4A) cells. Cells were cultured in 96-well plates, under normoxia or hypoxia, and treated with 2DG (8 mM) and STX140 (0.1 μM-1 μM) for 3 days when their effects on ATP were measured using the microtiter, ATPlite plate assay. A two-tailed Student's t-test assuming unequal variance was carried out, comparing values to normoxic STX140 untreated controls (black) or to STX140 treated compared to normoxic untreated controls (grey). ns=not significant; *p<0.05; **p<0.01; ***p<0.001. FIG. 4C shows percentage ATP change/live cell equivalent after 72 h in LNCaP & MCF-7 cells in response to 2DG alone. A one-way ANOVA test was carried out. Comparing 2DG treated cells to untreated cells. LNCaP; n=3, MCF-7; n=2 *p<0.05; ***p<0.001}

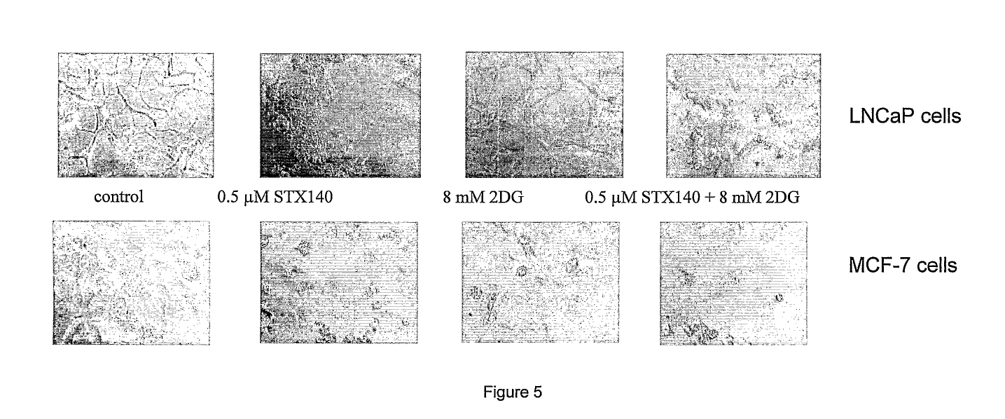

FIG. 5 shows: Morphology change in MCF-7 or LNCaP cells treated with compounds for 96 h, under normoxia.

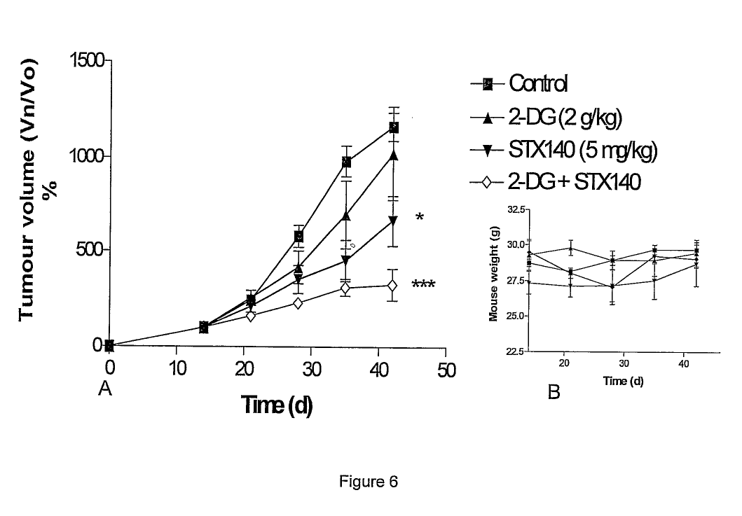

FIG. 6 shows: Growth of MCF-7 tumours in athymic nude mice. Growth of MCF-7 tumours (A) was inhibited by STX140 (5 mg/kg) (p<0.05) and STX140 (5 mg/kg)+2DG (2 g/kg) (p<0.01). There was no effect on mouse weight throughout the study (B) indicating a lack of compound toxicity. Points, mean percentage change in tumour volume (n=2-5 tumours); bars, SE, *p<0.05; **p<0.01 compared with control.

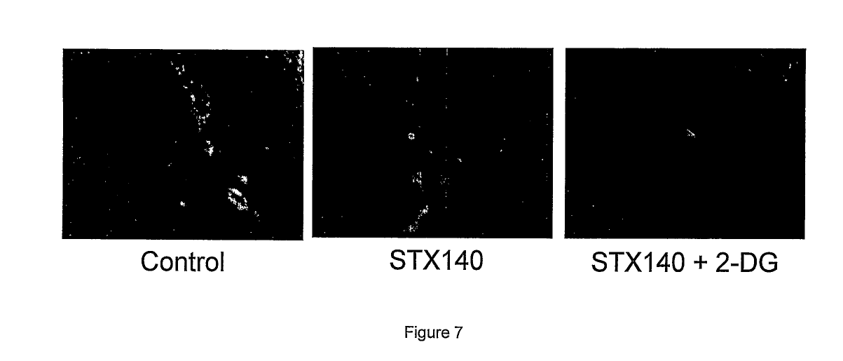

FIG. 7 shows: Von Willebrand's factor staining of blood vessels. Administration of STX140 (5 mg/kg p.o.; daily) or STX140 (5 mg/kg p.o.; daily)+2DG (2 g/kg i.p.; daily) caused a decrease in blood vessels relative to tumours taken from untreated animals. Magnification ×200.

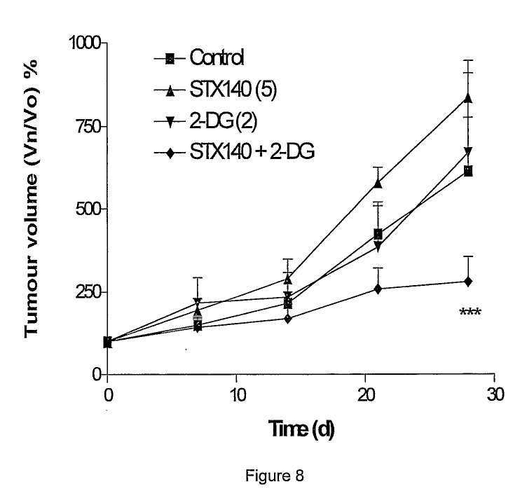

FIG. 8 shows: Growth of LNCaP tumours in athymic nude mice. Growth of LNCaP tumours was inhibited by STX140 (5 mg/kg)+2DG (2 g/kg) (p<0.001).

The present invention will now be described only by way of example. However, it is to be understood that the examples also present preferred compounds of the present invention, as well as preferred routes for making same and useful intermediates in the preparation of same.

Materials and Methods

Compounds

2-MeOE2bisMATE (STX140) was synthesised at the Department of Pharmacy and

Pharmacology, University of Bath (Leese et al., 2006). The compound displayed spectroscopic and analytical data in accordance with structure. 2-DEOXY_{-D-}GLUCOSE was obtained from Sigma (Irvine, England).

Cell Culture

MCF-7 and LNCaP cells were obtained from the American Tissue Culture Collection (ATCC). Cells were cultured in the following supplements, obtained from Sigma: RPMI 1640 with 10% foetal bovine serum, 1% L-glutamine, 1% MEM non-essential amino acids, 1% sodium bicarbonate solution, LNCaP cells also required 1% sodium pyruvate. Cells were maintained in an incubator at 37° C. under 5% CO₂ and 95% air atmosphere.

Proliferation Assay

MCF-7 and LNCaP cells were seeded at 3000 cells/well or 5000 cells/well respectively, in 96 well plates. Compounds were added 4 hours after seeding of cells. Alamar blue 10 ml (Biosource, Nivelles, Belgium) was added and cell proliferation was measured 72 hrs later using a FluoStar Optima Plate reader.

Hypoxia Model

An Innova co-48 CO₂ humidified incubator (Newbrunswick Scientific, St. Albans, U.K.) was used to study the effects of 2DG and STX140 on cells under hypoxic conditions. Oxygen was maintained at 1%, CO₂ 5% and temperature at 37° C.

Measurement of ATP

MCF-7 and LNCaP cells were seeded at 3000 cells/well or 5000 cells/well respectively, in 96 well plates. Compounds were added 4 hours following seeding of cells. The ATPlite assay procedure was carried out according to manufacturer's instructions (Perkin Elmer, Beaconsfield, U.K.) and ATP measured 72 hrs later using a FluoStar Optima Plate reader.

Photography

Cells were examined using a Zeiss inverted microscope fitted with a 20× and 40× Plan-Neofluor objective, and analysed through an Axiovision imaging system attached to the microscope.

Immunoblotting

Immunoblotting was performed as previously described (Newman et al., 2004). The following antibodies were used: Anti-PAMPK;1: 1000 dilution (Millipore, Chanders Ford, U.K.), Anti-PBCL-2; 1:100 dilution (Cell Signaling, MA, U.S.A.), Anti-PP70S6K; 1:1000 dilution (Cell Signaling), Anti-phoshpo-SAPK/JNK; 1:1000 dilution (Cell Signaling), Anti-p21; 1:500 dilution (BD Biosciences, Oxford, U.K.) or anti-beta-actin; 1:3000 dilution (Abcam, Cambridge, U.K.) primary antibody, the wash was then repeated and the secondary antibodies added which were either anti-rabbit; 1:5000 dilution (Cell Signaling), anti-mouse; 1:5000 dilution (Cell Signaling) or anti-goat; 1: 5000 dilution (Abcam). After thoroughly washing the membrane it was developed using ECF substrate (Amersham Biosciences, Little Chalfont, U.K.) and analysed using a STORM Optical Scanner. Whole cell lysate was loaded onto the immunoblot, a Bradford assay was used to assess the protein content of the nuclear extracts to ensure equal protein loading in each lane.

In Vivo Tumour Xenograft Model

MCF-7 Cell Tumours

Intact, ovariectomized, athymic, female MF-1 nude mice (nu-/nu-) were purchased from Harlan (Bicester, Oxon, UK) at 5 weeks of age (˜20-25 g in weight). All experiments were carried out under conditions that complied with institutional guidelines. Animals were kept in a 12-hour light/12-hour dark cycle and given food and water ad libitum. Monolayers of MCF-7 cells were removed by trypsinization, and the resultant cell suspension was centrifuged for 5 min. at 1,000×g and then resuspended in ice-cold matrigel (BD Biosciences). Five million MCF-7 cells were injected s.c. into the right flank of the animals. The tumours were allowed to grow for 2 weeks, when the tumours reached 100-150 mm³ in volume, mice were randomly divided into four treatment groups: 1) vehicle (10% tetrahydrofuran; 90% propylene glycol, 100 ml oral)+saline i.p. 200 ml; 2) vehicle+2DG (2 g/kg) i.p. 200 ml; 3) STX140 (5 mg/kg) 100 ml oral+saline i.p. 200 ml; 4) STX140 (5 mg/kg) 100 ml oral+2DG (2 g/kg) i.p. 200 ml. All treatments were administered daily for 4 weeks, 2DG was given i.p. (100 ml) and STX140 or vehicle was given orally (200 ml). Throughout the study, mice were weighed and tumour measurements were taken on a weekly basis. Tumour volumes were calculated using the formula: length×width²/2. At the end of the study, tumours were collected for immunohistochemical analysis.

LNCaP Cell Tumours

Athymic, male MF-1 nude mice (nu-/nu-) were purchased from Harlan (Bicester, Oxon,

UK) at 5 weeks of age (˜20-25 g in weight). All experiments were carried out under conditions that complied with institutional guidelines. Animals were kept in a 12-hour light/12-hour dark cycle and given food and water ad libitum. Monolayers of LNCaP cells were removed by trypsinization, and the resultant cell suspension was centrifuged for 5 min. at 1,000×g and then resuspended in ice-cold matrigel (BD Biosciences). Five million LNCaP cells were injected s.c. into the right flank of the animals. The tumours were allowed to grow for 3 weeks, when the tumours reached 100-150 mm³ in volume, mice were randomly divided into four treatment groups: 1) vehicle (10% tetrahydrofuran; 90% propylene glycol, 100 μl p.o.+saline i.p. 200 μl; 2) vehicle p.o.+2DG (2 g/kg) i.p.+200 μl; 3) STX140 (5 mg/kg) 100 μl p.o.+saline i.p. 200 μl; 4) STX140 (5 mg/kg) 100 μl p.o.+2DG (2 g/kg) i.p. 200 μl. All treatments were administered 5 days per week for 4 weeks, 2DG was given i.p. (200 μl) and STX140 or vehicle was given orally (100 μl). Throughout the study, mice were weighed and tumour measurements were taken on a weekly basis. Tumour volumes were calculated using the formula: length x width²/2.

Immunohistochemistry

von Willebrand's factor IHC were performed on paraffin embedded MDA-MB-231 tumor sections cut at 6 mm. After sectioning, rehydration and antigen retrieval steps, von Willebrand antibody (1:800, Abcam, Cambridge, United Kingdom) was applied to the section for 1 h at RT, followed by a goat polyclonal secondary antibody conjugated to FITC (30 min at RT). Sections were then mounted and viewed under a light or fluorescence microscope.

Statistics

All experiments were carried out in triplicate and data presented are representative of one of three such experiments, unless indicated. All errors shown are the mean±SE. Student's t test was used to assess the significance of the differences in cell proliferation for all experiments and ANOVA was used to look at the percentage change in ATP per live cell equivalent.

Results

Cell Proliferation Assays: Effect of 2DG Alone.

The ability of 2DG to inhibit the proliferation of LNCaP and MCF-7 cell lines was examined over a 5 day period under normoxic conditions (FIG. 2). The graph clearly shows a reduction in MCF-7 and LNCaP cell proliferation with increased 2DG concentration. The potency of 2DG was similar in both cell lines (MCF-7; IC₅₀: 8.1 mM and LNCaP; IC₅₀: 6.7 mM). The differences in IC₅₀ between MCF-7 and LNCaP cells were not significant. Several groups have carried out experiments using 2DG at concentrations between 1-24 mM (Aft et al., 2002; Kaplan et al., 1990; Lampidis et al., 2006; Zhu et al., 2005) and based on these findings, 8 mM 2DG was used throughout the study.

Cell Proliferation Assays: Effect of 2DG and STX140, Alone and in Combination.

The growth inhibitory effects of 2DG and STX140, used alone and in combination were compared (FIG. 3). Two cell lines were utilised, under normoxic or hypoxic conditions. The growth inhibition was determined after 72 h. Compared to normoxic untreated controls, STX140 (0.5 μM) inhibited cell proliferation by 65% in LNCaP cells (p<0.001, FIG. 3b) both under normoxia and hypoxia and by 45% and 48% in MCF-7 cells (p<0.01, FIG. 3a) under normoxia and hypoxia respectively. The IC₅₀ was calculated in LNCaP cells, both in normoxic (293 nM) and hypoxic (316 nM) conditions. These IC₅₀ values were not significantly different, and similar values have previously been calculated by Day and Newman (MCF-7: IC₅₀; 250 nM and LNCaP: IC₅₀; 260 nM) (Day et al., 2003; Newman et al., 2004). Under normoxic conditions, in both cell types, 2DG alone (8 mM) inhibited cell growth by 50% (LNCaP: p<0.01 and MCF-7: p<0.001). Hypoxic conditions further decreased cell growth to 70% (MCF-7: p<0.001) and 75% (LNCaP: p<0.001). Compared to STX140 at 0.5 μM, 2DG (8 mM) is significantly more effective at inhibiting tumour cell growth in both cell types (MCF-7: p<0.01 and LNCaP: p<0.05) under hypoxia. The ability of STX140 and 2DG combined to inhibit cell proliferation was studied in both cell types. In LNCaP cells the combination of 2DG and STX140 significantly decreased cell number, whether in normoxia or hypoxic conditions, compared to normoxic, untreated control. Under normoxic conditions 2DG (8 mM) reduces cell proliferation by 50%, this is increased to 60% (p<0.05) at 0.1 pM STX140, 68% (p<0.001) at 0.5 μM STX140 and 70% (p<0.001) at 1μ STX140.

Under hypoxic conditions, 2DG alone reduces the proliferation of LNCaP cells by 75%, this inhibition is further decreased to 85% (p<0.05) with addition of STX140 (0.5μ) down to 87% (p<0.01) with addition of STX140 (1μ).

Effect of 2DG and STX140 on ATP Levels

The ATP levels of MCF-7 and LNCaP treated with 2DG and STX140 were studied (FIG. 4a and b). Cells were incubated under normoxic or hypoxic conditions and ATP measured after 72 h. The concentration of ATP drops significantly in both cell types after treatment with STX140 (p<0.01-p<0.001) compared to untreated normoxic controls. 2DG used alone decreases ATP in normoxia (p<0.01) and hypoxia (p<0.001) compared to untreated normoxic controls. 2DG and STX140 reduce ATP levels at all concentrations of STX140 under normoxia (p<0.05-0.001) and hypoxia shows the greatest reduction in ATP (p<0.001) compared to untreated normoxic controls. The effect of 2DG on ATP concentration was calculated per living MCF-7 or LNCaP cell (FIG. 4c). 2DG (8 mM) significantly decreases ATP in living cells; LNCaP (p<0.05) and MCF-7 (p<0.001) compared to untreated controls. Karczmar et at showed tumour ATP reduction caused by 2DG (Karczmar et al., 1992).

Cell Morphology

Images show LNCaP and MCF-7 cells treated with STX140 (0.5 μM) and 2DG 8 mM) alone or in combination (FIG. 5). Untreated cells look healthy compared to the shrivelled appearance of STX140 treated cells. When 2DG is added the cells appear to swell up, possibly due to 2DG being trapped in the cell and osmosis occurring. The cells look less healthy when the compounds are combined, with shrivelled, swollen apoptotic-looking cells.

Effect of STX140 and 2DG In Vivo

### MCF-7 Cell Tumours

Following inoculation, MCF-7 cells developed into stable growing tumours, with a “take rate” of 80%. Once tumours had reached approximately 100-150 mm³ in volume, compound dosing commenced for 28 days. At the end of dosing growth of the MCF-7 tumours (FIG. 6a) was significantly inhibited by STX140 (5 mg/kg p.o.; daily) (p<0.05) compared to control. 2DG (2 g/kg i.p.; daily) alone did not significantly inhibit tumour growth, compared to control. However, the combination of STX140 (5 mg/kg p.o.; daily) and 2DG (2 g/kg i.p.; daily) did significantly inhibit tumour proliferation (p<0.001). FIG. 6b reveals the changes in the weights of mice throughout the study. No weight loss occurred, indicating that the animals tolerated the compounds without any toxicity.

Expression of von Willebrand's Factor

The endothelial specific marker, von Willebrand's factor, was used to assess vessel density in sections taken from MCF-7 xenografts taken from treated animals. FIG. 7 shows both STX140 (5 mg/kg p.o.; daily) and STX140 (5 mg/kg p.o.; daily) combined with 2DG (2 g/kg i.p.; daily) caused a significant reduction in the staining for endothelial cells. 2DG (2 g/kg i.p.; daily) alone had no significant effect (data not shown).

LNCaP Cell Tumours

Once tumours had reached approximately 100-150 mm³ in volume, compound dosing commenced for 5 days per week for 4 weeks. At the end of dosing growth of the LNCaP tumours was significantly inhibited by the combination of STX140 (5 mg/kg p.o.) and 2DG (2 g/kg i.p.) (p<0.001).

Discussion

The aim of this study was to improve inhibition of tumour cell proliferation using the combination of an alkoxy/sulphamate substituted ring system compound and a glycolytic inhibitor in vitro and in vivo. Tumour cells, even in the presence of oxygen, continue to rely on glycolysis rather than oxidative phosphorylation (Warburg, 1956). Several oncogenes have been implicated in the Warburg effect; the AKT oncogene is associated with enhanced glucose uptake and aerobic glycolysis, independent of HIF-1 (Elstrom et al., 2004). AKT assembles glucose transporters to the cell surface to enhance glucose uptake and activate hexokinase II (HK2) to phosphorylate and trap intracellular glucose. AKT is able to enhance glycolytic flux without affecting mitochondrial oxidative phosphorylation, contributing to the Warburg effect. The MYC oncogene activates virtually all glycolytic enzyme genes, including those encoding HK2, enolase, and lactate dehydrogenase A (LDHA) (Kim & Dang, 2005). The inner core of tumour cells are particularly resistant to many of the anti-cancer agents which target rapidly dividing cells (Liu et al., 2001).

Anaerobically metabolizing, slow growing tumour cells demonstrate another form of multidrug resistance (MDR) in addition to the MDR mechanisms previously identified, for example the ABC cassette family of transporter proteins, P-GP and MRP (Hendrikse, 2000; Kuwano et al., 1999) and topoisomerase (Fortune & Osheroff, 2000; Gupta et al., 2005).

STX140 causes caspase-dependent apoptosis in CAL51 breast cancer cells and overcomes resistance to TRAIL by activating caspases (Wood et al., 2004). STX140 has been shown to be 6 times more potent an inhibitor of breast cancer cells compared to 2-MeOE2 (Raobaikady et al., 2003) and can inhibit proliferation of HUVEC cells 60 fold more effectively than 2-MeOE2 and was 10-13 times more active as an inhibitor of vessel formation in a novel co-culture model system (Newman et al., 2004). STX140 was also effective in cells resistant to mitoxantrone or doxorubicin (Suzuki et al., 2003a) and was also a very active anti-tumour agent in vivo (Ireson et al., 2004).

STX140 also has good STS inhibitory properties in vitro and in vivo (Raobaikady et al., 2003) which is beneficial for the treatment of hormone dependent breast cancers. STX140 shows enhanced oral availability and improved pharmacokinetic properties (Ireson et al., 2004). Ho et al., showed that TNF-aand STX140 used together increased the potency of 2-substituted oestrogens as anti-angiogenic agents via synergistic induction of apoptosis in endothelial cells and had low cytotoxicity in fibroblasts (Ho et al., 2003). Fluorescent images of MDA-MB-231 cells treated with STX140 revealed disintegrated tubulin, condensed nuclei and microtubule damage. In addition, STX140 caused cell cycle arrest in MDA-MB-231 cells (Raobaikady et al., 2005). To summarise the anti-tumour affects of STX140 are caused by disruption of microtubules leading to cell cycle arrest and subsequent apoptosis. Furthermore, STX140 has potential in vitro and in vivo anti-angiogenic activity (Newman 2004, Chander 2007[submitted] and Foster et al., 2007 [submitted]).

The results from this study confirm that 2DG is a potent inhibitor of both LNCaP and MCF-7 cell proliferation, with increased potency under hypoxic conditions. Other groups have also showed 2DG to be very efficacious at inhibiting tumour cell growth (Lyon et al., 1988 and Kaplan et al., 1990).

In addition, Liu et al used two models to investigate the tumour cells dependency on glycolysis. The first represented osteosarcoma wild-type (wt) cells treated with agents which inhibit mitochondrial oxidative phosphorylation (Oxphos) by interacting with complexes I, III and V of the electron transport chain in different ways, inhibitors included

Rhodamine (Rho 123), rotenone, antimycin A and oligomycin. All of these Oxphos inhibitors were found to hypersensitize wild-type cells to 2DG. Cells treated with Rho 123 also became hypersensitive to oxamate, an analogue of pyruvate which blocks the step of glycolysis that converts pyruvate to lactic acid. The second model is r^{□} cells which have lost their mitochondrial DNA and therefore cannot undergo Oxphos. These cells were 10 and 4.9 times more sensitive to 2DG than oxamate, respectively, than wt cells. Overall, Liu et at showed that the glycolytic inhibitors oxamate and 2DG could be used to specifically target the slow-growing cells of a tumour and thereby increase the efficacy of current chemotherapeutic and irradiation protocols designed to kill rapidly dividing cells. They also hypothesised that glycolytic inhibitors could be particularly useful in combination with anti-angiogenic agents which should make tumours more anaerobic and therefore, reliant on glycoloysis (Liu et al., 2001).

Aft et al studied the effects of 2DG on breast cancer cells in vitro: 2DG treatment of breast cancer cells resulted in cessation of cell growth in a dose-dependent manner. Cell death was induced by 2DG caused by apoptosis, with induction of caspase 3 activity and cleavage of poly (ADP-ribose) polymerase. GLUT 1 transporter was elevated and glucose uptake elevated compared to non-2DG treated breast cancer cells. It was concluded that 2DG causes death in human breast cancer cells by activation of the apoptotic pathway, they accelerate their own demise by initially expressing high levels of glucose transporter protein, which allowed increased uptake of 2DG and subsequent induction of cell death. Aft et al revealed good evidence to support the targeting of glucose metabolism as a site for chemotherapeutic intervention by agents such as 2DG (Aft et al., 2002).

The results of this study revealed how 2DG depletes ATP in vitro. Xu et at showed how inhibition of glycolysis severely depleted ATP in cancer cells, especially those cells with mitochondrial defects and led to rapid dephosphorylation of the glycolysis-apoptosis interacting molecule BAD at Ser¹¹², relocalization of BAX to mitochondria and massive cell death. 2DG was shown to utilise a novel strategy to overcome drug resistance associated with mitochondrial respiratory defect and hypoxia (Xu et al., 2005).

Zhu et al showed in MCF-7 cells that 2DG reduced cell growth and intracellular ATP in a dose- and time-dependent manner. 2DG increased levels of phosphorylated AMPK and Sirt-1 and reduced phosphorylation of Akt. This study supported the hypothesis that dietary energy restriction (DER) inhibits carcinogenesis, partly by limiting glucose availability and that energy metabolism is a target for the development of energy restriction mimetic agents for chemoprevention (Zhu et al., 2005). Recently, Lampidis et at created 2-halogenated D-glucose analogs for improved activity, compared to 2DG. 2-fluor-2-deoxy-D-glucose (2FG) was more potent than 2DG in killing hypoxic tumour cells and would therefore could be more clinically effective when combined with standeard chemotherapeutic protocols (Lampidis et al., 2006).

The results of this study revealed how there is significant improvement in cell proliferation inhibition when a glycolytic inhibitor (such as 2DG) is combined with a alkoxy/sulphamate substituted ring system compound (such as STX140) in LNCaP cells. The combination of compounds was not more effective than either agent alone in MCF-7 cells in vitro. The in vitro proliferation assay used in these studies does not reflect the in vivo situation; there is no inner core of slow growing tumour cells that would be potentially recalcitrant to STX140 but sensitive to 2DG. The true benefit of combining agents with 2DG may only become apparent using in vivo tumour models.

The combination of 2DG and STX140 is the most effective at inhibiting tumour xenograft growth compared to either agent alone, in vivo. The dose of STX140 used in vivo was only 5 mg/kg, it would be possible to increase the efficiency of STX140 by increasing the dose to 20 mg/kg as used by Foster et al (Foster et al., 2006[submitted]).

Several groups have also showed beneficial effect when chemotherapeutic agents are combined with 2DG, with the hope of targeting the aerobic rapidly dividing and anaerobic, slowly dividing cells: Maschek et al utilised 2DG in combination with Adriamycin (ADR) or Paclitaxel in nude mouse xenograft models of human osteosarcoma and non-small cell lung cancer. Nude mice were implanted with osterosarcoma cells and divided into four groups: 1) untreated, 2) ADR alone, 3) 2DG alone, 4) ADR+2DG. Tumours were 50 mm³ or 300 mm³ in volume when treatment began. Starting with small or large tumours, the ADR+2DG combinations resulted in significantly slower tumour growth (and therefore longer survival) then the control, 2DG or ADR treatments (p<0.0001). Similar beneficial effects of combination treatment were found with 2DG and paclitaxel in MV522 non-small lung cancer xenograft model. Their results provided a rationale for initiating clinical trials using glycolytic inhibitors in combination with chemotherapeutic agents to increase their therapeutic effectiveness (Maschek et al., 2004). In 2005, Gupta et al showed how 2DG enhances the efficacy of etoposide in Ehrlich ascites tumour-bearing mice. Analysis of cells obtained from ascitic fluid as well as solid tumours during follow up-revealed etoposide induced cell death was mainly due to apoptosis, which was enhanced further by 2DG. In addition, 2DG did not increase the toxicity of etoposide at 60 mg/kg. Therefore, administration of 2DG can improve the local control of tumours without increasing normal tissue toxicity, so enhancing the therapeutic efficacy of topoisomerase inhibitor -based anti-cancer drugs like etoposide (Gupta et al., 2005).

The results of this study revealed how STX140 causes a decrease in von Willebrand's factor staining, thus supporting the anti-angiogenic potential of this compound observed in vitro (Newman et al., 2004). The combination of STX140 and 2DG was the most efficacious at inhibiting in vivo tumour growth, this supports the hypothesis that the present anti-angiogenic agents may potentiate the activity of 2DG due to an increase in hypoxia and greater reliance on glycolysis.

Raez et al have instigated a Phase 1 trial of 2DG and cocetaxel in patients with solid tumours and the drug combination was shown to be feasible and safe (Raez et al., 2005)

In conclusion, both STX140 and 2DG are very potent anti-cancer compounds in vitro and in vivo. There are clear advantages of using multi-targeting treatments, combining anti-angiogenic activity, microtubule disruption with 2DG to attack the whole of the tumour. The anti-angiogenic properties of STX140 results in less blood vessels, an increase in hypoxia and therefore more glycolysis, thus sensitizing the cell to 2DG. The microtubule disruption of STX140 will target rapidly dividing cells, inhibiting cell proliferation. Using multiple compounds reduces the likelihood of resistance so lower doses of microtubule disruptors can be used causing less off target effects. In the future it may be very beneficial to modify 2DG in order to make it more efficacious in cancer prevention.

All publications and patents mentioned in the above specification are herein incorporated by reference.

Various modifications and variations of the present invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry, biology or related fields are intended to be within the scope of the following claims.

### REFERENCES

1. Aft, R. L., Zhang, F. W. & Gius, D. (2002). Evaluation of 2-deoxy-D-glucose as a chemotherapeutic agent: mechanism of cell death. Br J Cancer, 87, 805-12.
2. Aloj, L., Caraco, C., Jagoda, E., Eckelman, W. C. & Neumann, R. D. (1999). Glut-1 and hexokinase expression: relationship with 2-fluoro-2-deoxy-D-glucose uptake in A431 and T47D cells in culture. Cancer Res, 59, 4709-14.
3. Arora, K. K., Parry, D. M. & Pedersen, P. L. (1992). Hexokinase receptors: preferential enzyme binding in normal cells to nonmitochondrial sites and in transformed cells to mitochondrial sites. J Bioenerg Biomembr, 24, 47-53.

4. Attalla, H., Westberg, J. A., Andersson, L. C., Aldercreutz, H. & Makela, T. P. (1998). 2-Methoxyestradiol-induced phoshphorylation of Bcl-2: Uncoupling from JNK/SAPK activationI. Biochem Biophys Res Commun, 247, 616-619.
5. Basu, A. & Haldar, S. (2003). Identification of a novel Bcl-xL phosphorylation site regulating the sensitivity of taxol- or 2-methoxyestradiol-induced apoptosis. FEBS Lett, 538, 41-47.
6. Birnbaum, M. J., Haspel, H. C. & Rosen, O. M. (1987). Transformation of rat fibroblasts by FSV rapidly increases glucose transporter gene transcription. Science, 235, 1495-8.
7. Boyle, P. & Ferlay, J. (2005). Cancer incidence and mortality in Europe, 2004. Annals of Oncology, 1-8.
8. Carew, J. S. & Huang, P. (2002). Mitochondrial defects in cancer. Mol Cancer, 1, 9.
9. Carew, J. S., Zhou, Y., Albitar, M., Carew, J. D., Keating, M. J. & Huang, P. (2003). Mitochondrial DNA mutations in primary leukemia cells after chemotherapy: clinical significance and therapeutic implications. Leukemia, 17, 1437-47.

10. Carothers, A. M., Hughes, S. A., Ortega, D. & Bertagnolli, M. M. (2002). 2-Methoxyestradiol induces p53-associated apoptosis of colorectoral cancer cells. Cancer Lett, 187, 77-86.
11. Chauhan, D., Catley, L., Hideshima, T. & al, e. (2002). 2-Methoxyestradiol overcomes drug resistance in multiple myeloma cells. Blood, 100, 2187-2194.
12. Cohen, P. (1997). The search for physiological substrates of MAP and SAP kinases in mammalian cells. Trends Cell Biol., 7, 353-361.
13. D'Amato, R. J., Lin, C. M., Flynn, E., Folman, J. & Hamel, E. (1994). 2-Methoxyestradiol, an endogenous mammalian metabolite, inhibits tubulin polymerization by interacting at the colchicine site. Proc Natl Acad Sci U S A, 91, 3964-3968.
14. Day, J. M., Newman, S. P., Comninos, A., Solomon, C., Purohit, A., Leese, M. P., Potter, B. V. & Reed, M. J. (2003). The effects of 2-substituted oestrogen sulphamates on the growth of prostate and ovarian cancer cells. J Steroid Biochem Mol Biol, 84, 317-25.
15. Dennis, P. B., Jaeschke, A., Saitoh, M., Fowler, B., Kozma, S. C. & Thomas, G. (2001). Mammalian TOR: a homeostatic ATP sensor. Science, 294, 1102-1105.
16. Elstrom, R. L., Bauer, D. E., Buzzal, M. & al, e. (2004). Akt stimualtes aerobic glycolysis in cancer cells. Cancer Res, 64, 3892-9.
17. Flier, J. S., Mueckler, M. M., Usher, P. & Lodish, H. F. (1987). Elevated levels of glucose transport and transporter messenger RNA are induced by ras or src oncogenes. Science, 235, 1492-5.
18, Fortune, J. M. & Osheroff, N. (2000). Topoisomerase II as a target for anticancer drugs: when enzymes stop being nice. Prog Nucleic Acid Res Mol Biol, 64, 221-53.
19. Foster, P. A., Newman, S. P., Chander, S. K., Stengal, C., Jhalli, R., Woo, L. L., Potter, B. V., Reed, M. J. & Purohit, A. (2006). In vivo Efficacy of STX213, A Second-Generation Steroid Sulfatase Inhibitor, for Hormone-Dependent Breast Cancer Therapy. Clin Cancer Res, 12, 5543-5549.
20. Fotsis, T., Zhang, Y., Pepper, M. S., Adlercreutz, H., Montesano, R., Nawroth, P. P. & Schweigerer, L. (1994). The endogenous oestrogen metabolite 2-methoxyoestradiol inhibits angiogenesis and suppresses tumour growth. Nature, 368, 237-9.
21. Fryer, L. G., Foufelle, F., Barnes, K., Baldwin, S. A., Woods, A. & Carling, D. (2002).

Characterization of the role of the AMP-activated protein kinase in the stimulation of glucose transport in skeletal muscle cells. Biochem J, 363, 167-74.
22. Gallagher, B. M., Fowler, J. S., Gutterson, N. I., MacGregor, R. R., Wan, C. N. & Wolf, A. P. (1978). Metabolic trapping as a principle of oradiopharmaceutical design: some factors resposible for the biodistribution of [18F] 2-deoxy-2-fluoro-D-glucose. J Nucl Med, 19, 1154-61.
23. Geschwind, J. F., Georgiades, C. S., Ko, Y. H. & Pedersen, P. L. (2004). Recently elucidated energy catabolism pathways provide opportunities for novel treatments in hepatocellular carcinoma. Expert Rev Anticancer Ther, 4, 449-57.
24. Godov, A., Ulloa, V., Rodriquez, F., Reinicke, K., Yanez, A. J., Garcia Mde, L., Carrasco, M., Barberis, S., Castro, T., Martinez, F., Koch, X., Vera, J. C., Poblete, M. T., Figueroa, C. D., Peruzzo, B., Perez, F. & Nualart, F. (2006). Differential subcellular distribution of glucose transporters GLUT1-6 and GLUT9 in human cancer: untrastructural localization of GLUT1 and GLUT5 in breast tumor tissues. Journal of Cell Physiology, 207, 614-27.
25. Goldman, N. A., Katz, E. B., Glenn, A S., Weldon, R. H., Jones, J. G., Lynch, U., Fezzari, M. J., Runowicz, C D., Goldberg, G. L. & Charron, M. J. (2006). GLUT1 and GLUT8 in endometrium and endometrial adenocarcinoma. Mod Pathol.
26. Gottesman, M. M., Fojo, T. & Bates, S. E. (2002). Multidrug resistance in cancer: Role of ATP dependent transporters. Nature, 2, 48-58.
27. Gupta, S., Mathur, R. & Dwarakanath, B. S. (2005). The Glycolytic inhibitor 2-Deoxy-D-Glucose Enhances the Efficacy of Etoposide in Ehrlich Ascites Tumour-Bearing Mice. Cancer Biology & Therapy, 4, 87-94.
28. Hardie, D. G. & Carling, D. (1997). The AMP-activated protein kinase: fuel gauge of the mammalian cell. Eur J Biochem, 246, 259-273.
29. Hardie, D. G., Carling, D. & Carlson, M. (1998). The AMP-activated/SNF1 protein kinase subfamily: metabolic sensors of the eukaryotic cell? Annu. Rev. Biochem., 67, 821-855.
30. Hardie, D. G., Salt, L P., Hawley, S. A. & Davies, S. P. (1999). AMP-activated protein kinase: an ultrasensitive system for monitoring cellular energy charge. Biochem. 388, 717-722.
31. Hendrikse, N. H. (2000). Monitoring interactions at ATP-dependent drug efflux pumps. Curr Pharm Des., 6, 1653-68.
32. Ho, Y. T., Newman, S. P., Purohit, A., Leese, M. P., Potter, B. V. & Reed, M. J. (2003). The effects of 2-methoxy oestrogens and their sulphamoylated derivatives in conjunction with TNF-alpha on endothelial and fibroblast cell growth, morphology and apoptosis. Journal of Steroid Biochemistry & Molecular Biology, 86, 189-196.
33. Huang, P., Feng, L., Oldham, E. A., Keating, M. J. & Plunkett, W. (2000). Superoxide dismutase as a target for the selective killing of cancer cells.[see comment]. Nature, 407, 390-5.
34. Inoki, K., Zhu, T. & Guan, K. L. (2003). TSC2 Mediates Cellular Engergy REsponse to

Control Cell Growth and Survival. Cell, 115, 577-590.
35. Ireson, C. R., Chander, S. K., Purohit, A., Perera, S., Newman, S. P., Parish, D., Leese, M. P., Smith, A. C., Potter, B. V. & Reed, M. J. (2004). Pharmacokinetics and efficacy of 2-methoxyoestradiol and 2-methoxyoestradiol-bis-sulphamate in vivo in rodents. British Journal of Cancer, 90, 932-7.
36. Jain, V. K., Kalia, V. K., Sharma, R., Maharajan, V. & Menon, M. (1985). Effects of 2-deoxy-D-glucose on glycolysis, proliferation kinetics and radiation response of human cancer cells. Int J Radiat Oncol Biol Phys, 11, 943-50.
37. Jordan, M. A. & Wilson, L. (1998). Microtubules and actin filaments: dynamic targets for cancer chemotherapy. Curr Opin Cell Biol, 10, 123-130.
38. Kalir, T., Wang, B. Y., Goldfischer, M., Haber, R. W. & al, e. (2002).

Immunohistochemical staining of GLUT1 in benign, borderline, and malignant ovarian epithelia. Cancer, 94, 1078-1082.
39. Kaplan, O., Navon, G., Lyon, R. C., Faustino, P. J., Straka, E. J. & Cohen, J. S. (1990). Effects of 2-deoxyglucose on drug-sensitive and drug-resistant human breast cancer cells: toxicity and magnetic resonance spectroscopy studies of metabolism. Cancer Res, 50, 544-51.
40. Karczmar, G. S., Arbeit, J. M., Toy, B. J. & Weiner, M. W. (1992). Selective depletion of tumor ATP by 2-deoxyglucose and insulin, detected by [31]P magnetic resonance spectroscopy. Cancer Res, 52, 71-76.
41. Kemp, B. E., Mitchelhill, K. I., Stapleton, D., Michell, B. J., Chen, Z.-P. & Witters, L. A. (1999). Dealing with energy demand: the AMP-activated protein kinase. Trends Biochem. Sci, 24, 22-25.
42. Kern, K. A. & Norton, J. A. (1987). Inhibition of established rat fibrosarcoma growth by the glucose antagonist 2-deoxy-D-glucose. Surgery, 102, 380-5.
43. Kim, J. W. & Dang, C. V. (2005). Multifaceted roles of glycolytic enzymes. Trends Biochem. Sci., 30, 142-50.
44. Klauber, N., Parangi, S., Flynn, E., Hamel, E. & D'Amato, R. J. (1997). Inhibition of angiogenesis and breast cancer in mice by the microtubule inhibitors 2-methoxyestradiol and taxol. Cancer Res, 57, 81-86.
45. Koukourakis, M. I., Giatromanolaki, A., Polychronidis, A., Simopoulos, C., Gatter, K. C., Harris, A. L. & Sivridis, E. (2006). Endogenous markers of hypoxia/anaerobic metabolism and anemia in primary colorectal cancer. Cancer Sci, 97, 582-8.
46. Kumar, A. P., Garcia, G. E., Orsborn, J., Levin, V. A. & Slaga, T. J. (2003). 2-Methoxyestradiol interferes with NF kappa B transcriptional activity in primitive neuroectodermal brain tumors: implications for management. Carcinogenesis, 24, 209-16.
47. Kuwano, M., Toh, S., Uchiumi, T., Takano, H., Kohno, K. & Wada, M. (1999). Multidrug resistance-associated protein subfamily transporters and drug resistance. Anticancer Drug Des., 14, 123-31.
48. Kyriakis, J. M. & Avruch, J. (1996). Sounding the alarm: protein kinase cascades activated by stress and inflammation. J Biol Chem, 271, 24313-6.
49. La Vallee, T. M., Zhan, X. H., Herbstritt, C. J., Kough, E. C., Green, S. J. & Pribluda, V. S. (2002). 2-Methoxyestradiol inhibitis proliferation and induces apoptosis independantly of estrogen receptors alpha and beta. Cancer Res, 62, 3691-3697.
50. Lampidis, T., Kurtoglu, M., Maher, J. C., Liu, H., Krishan, A., Sheft, V., Szymanski, S., Fokt, I., Rundnicki, W. R., Ginalski, K., Lesyng, B. & Priebe, W. (2006). Efficacy of 2-halogen substituted D-glucose analogs in blocking glycolysis and killing “hypoxic tumor cells”. Cancer Chemother Pharmacol, 58, 725-734.
51. Laszlo, J., Humphreys, S. R. & Goldin, A. (1960). Effects of glucose analogues (2-deoxy-D-glucose, 2-deoxy-D-galactose) on experimental tumors. J Natl Cancer Inst, 24, 267-81.
52. Landau B R, Laszlo J, Stengle J, Burk D. Certain metabolic and pharmacologic effects in cancer patients given infusions of 2-deoxy-D-glucose. J Natl Cancer Inst. 1958 September;21(3):485-94.
53. LaVallee, T. M., Zhan, X. H., Johnson, M. S., Herbstritt, C. J., Swartz, G., Williams, M. S., Hembrough, W. A., Green, S. J. & Pribluda, V. S. (2003). 2-methoxyestradiol up-regulates death receptor 5 and induces apoptosis through activation of the extrinsic pathway. Cancer Research, 63, 468-75.
54. Lee, H. H., Dadgostar, H., Cheng, Q., Shu, J. & Cheng, G. (1999). NF-kappaB-mediated up-regulation of Bcl-x and Bfl-1/A1 is required for CD40 survival signaling in B lymphocytes. Proceedings of the National Academy of Sciences of the United States of America, 96, 9136-41.
55. Leese M P, Leblond B, Smith A, at al. 2-substituted estradiol bis-sulfamates, multitargeted antitumor agents: synthesis, in vitro SAR, protein crystallography, and in vivo activity. J Med Chem. 2006;49(26):7683-96.
56. Liu, H., Hu, Y. P., Savaraj, N., Priebe, W. & Lampidis, T. J. (2001). Hypersensitization of tumor cells to glycolytic inhibitors. Biochemistry, 40, 5542-5547.
57. Lyon, R. C., Cohen, J. S., Faustion, P. J., Megnin, F. & Myers, C. E. (1988). Glucose metabolism in drug-sensitive human breast cancer cells monitored by magnetic resonance spectroscopy. Cancer Res, 48, 870-7.
58. Mabjeesh, N. J., Escuin, D., La Vallee, T. M. & al, e. (2003). 2ME2 inhibits tumor growth and angiogenesis by disrupting microtubules and dysregulating HIF. Cancer Cell, 3, 363-375.
59. MacCarthy-Morrogh, L., Townsend, P. A., Purohit, A., Hejaz, H. A. M., Potter, B. V. L., Reed, M. J. & Packham, G. (2000). Differential effects of estrone and estrone-3-O-sulfamate derivatives on mitotic. Arrest, apoptosis, and microtubule assembly in human breast cancer cells. Cancer Res, 60, 5441-50.
60. Marsin, A.-S., Bertrand, L., Rider, M. N., Deprez, J., Beauloye, C., Vincent, M.-F., Van den Berghe, G., Carling, D. & Hue, L. (2000). Curr Biol, 10, 1247-1255.
61. Maschek, G., Savaraj, N., Priebe, W., Braunschweiger, P., Hamilton, K., Tidmarsh, G. F., De Young, L. R. & Lampidis, T. J. (2004). 2-deoxy-D-glucose increases the efficacy of adriamycin and paclitaxel in human osteosarcoma and non-small cell lung cancers in vivo. Cancer Research, 64, 31-4.
62. Moley, K. H. & Mueckler, M. M. (2000). Glucose transport and apoptosis. Apoptosis, 5, 99-105.
63. Mukhopadhyay, T. & Roth, J. A. (1997). Induction of apoptosis in human lung cancer cells after wild-type p53 activation by methoxyestradiol. Oncogene., 14, 379-384.
64. Nelson, C. A., Wang, J. Q ., Leav, I. & Crane, P. D. (1996). The interaction among glucose transport, hexokinase, and glucose-6-phosphatase with respect to 3H-2-deoxyglucose retention in murine tumor models. Nucl Med Biol, 23, 533-41.
65. Newman, S. P., Leese, M. P., Purohit, A., James, D. R., Rennie, C. E., Potter, B. V. & Reed, M. J. (2004). Inhibition of in vitro angiogenesis by 2-methoxy- and 2-ethyl-estrogen sulfamates. International Journal of Cancer, 109, 533-40.
66. Noguchi, Y., Marat, D., Saito, A. & al, e. (1999). Expression of facilitative glucose transporters in gastric tumors. Hepatogastroenterology, 46, 2683-2689.
67. Pasqualini, J. R., Gelly, C., Nguyen, B. L. & Vella, C. (1989). Importance of estrogen sulfates in breast cancer. J Steroid Biochem, 34, 155-63.
68. Pelicano, H., Carney, D. & Huang, P. (2004). ROS stress in cancer cells and therapeutic implications. Drug Resist Updat, 7, 97-110.
69. Penta, J. S., Johnson, F. M., Wachsman, J. T. & Copeland, W. C. (2001). Mitochondrial DNA in human malignancy. Mutat Res, 488, 119-33.
70. Purohit, A., Froome, V. A., Wang, D. Y., Potter, B. V. & Reed, M. J. (1997). Measurement of oestrone sulphatase activity in white blood cells to monitor in vivo inhibition of steroid sulphatase activity by oestrone-3-O-sulphamate. J Steroid Biochem Mol Biol, 62, 45-51.
71. Purohit, A., Vernon, K. A., Hummelinck, A. E., Woo, L. W., Hejaz, H. A., Potter, B. V. & Reed, M. J. (1998). The development of A-ring modified analogues of oestrone-3-O-sulphamate as potent steroid sulphatase inhibitors with reduced oestrogenicity. J Steroid Biochem Mol Biol, 64, 269-75.
72. Purohit, A., Woo, L. W., Barrow, D., Hejaz, H. A., Nicholson, R. I., Potter, B. V. & Reed, M. J. (2001). Non-steroidal and steroidal sulfamates: new drugs for cancer therapy. Mol Cell Endocrinol, 171, 129-35.
73. Qin, S., Minami, Y., Hibi, M., Kurosaki, T. & Yamamura, H. (1997). Syk-dependent and -independent signaling cascades in B cells elicited by osmotic and oxidative stress. J Biol Chem, 272, 2098-103.
74. Raez, L. E., Rosenblatt, J., Schlesselman, J., Langmuir, V., Tidmarsh, G., Rocha-Lima, C., Papadopoulos, K., O'Connor, J., Baldie, P. & Lampidis, T. (2005). Combining glycolytic inhibitors with chemotherapy: Phase 1 trial of 2-deoxyglucose and docetaxel in patients with solid tumours. Journal of Clinical Oncology, 23, 3190.
75. Raobaikady, B., Purohit, A., Chander, S. K., Woo, L. W., Leese, M. P., Potter, B. V. & Reed, M. J. (2003). Inhibition of MCF-7 breast cancer cell proliferation and in vivo steroid sulphatase activity by 2-methoxyoestradiol-bis-sulphamate. J Steroid Biochem Mol Biol, 84, 351-8.
76. Raobaikady, B., Reed, M. J., Leese, M. P., Potter, B. V. & Purohit, A. (2005). Inhibition of MDA-MB-231 cell cycle progression and cell proliferation by C-2 substituted oestradiol mono- and bis-3-O-sulphamates. Int J Cancer, 117, 150-9.
77. Reed, M. J., Purohit, A., Woo, L. W. L. & Potter, B. V. L. (1996). The development of steroid sulphatase inhibitors. Endocr.-Re. Cancer, 3, 9-23.
78. Reiser, F., Way, D., Bernas, M., Witte, M. & Witte, C. (1998). Inhibition of normal and experimental angiotumour endothelial cell proliferation and cell cycle progression by 2-methoxyestradiol. Proc Soc Exp Biol Med, 219, 211-216.
79. Rivenzon-Segal, D., Rushkin, E., Polak-Charcon, S. & Degani, H. (2000). Glucose transporters and transport kinetics in retinoic acid-differentiated T47D human breast cancer cells. Am J Physiol Endocrinol Metab, 279, E508-19.

80. Rudlowski, C., Becker, A. J., Schroder, W. & al, e. (2003). GLUT1 mesenger RNA and protein induction relates to the malignant transformation of cervical cancer. Am J Clin Pathol, 120, 691-698.
81. Sattler, M., Quinnan, L. R., Pride, Y. B. & al, e. (2003). 2-Methoxyestradiol alters cell motility, migration, and adhesion. Blood, 102, 289-296.
82. Seegers, J. C., Aveling, M. L., Van Aswegen, C. H., Cross, M., Koch, F. & Joubert, W. S. (1989). The cytotoxic effects of estradiol-17 beta, catecholestradiols and methoxyestradiols on dividing MCF-7 and HeLa cells. J Steroid Biochem, 32, 797-809.
83. Seegers, J. C., Lottering, M. L., Grobler, C. J. & al, e. (1997). The mammalian metabolite, 2-methoxyestradiol, affects p53 levels and apoptosis induction in transformed cells but not in normal cells. J Steroid Bochem Mol Biol, 62, 253-267.
84. Suzuki, R. N., Newman, S. P., Purohit, A., Leese, M. P., Potter, B. V. & Reed, M. J. (2003a). Growth inhibition of multi-drug-resistant breast cancer cells by 2-methoxyoestradiol-bis-sulphamate and 2-ethyloestradiol-bis-sulphamate. Journal of Steroid Biochemistry & Molecular Biology, 84, 269-78.
85. Suzuki, T., Moriya, T., Ishida, T., Ohuchi, N. & Sasano, H. (2003b). Intracrine mechanism of estrogen synthesis in breast cancer. Biomed Pharmacother, 57, 460-2.
86. Van der Kaay, J., Beck, M., Gray, A. & Downes, C. P. (1999). Distinct phosphatidylinositol 3-kinase lipid products accumulate upon oxidative and osmotic stress and lead to different cellular responses. J Biol Chem, 274, 35963-8.
87. Vousden, K. H. & Lu, X. (2002). Live or let die: the cell's response to p 53. Nature Reviews. Cancer, 2, 594-604.
88. Waki, A., Fujibayashi, Y., Yonekura, Y., Sadato, N., Ishii, Y. & Yokoyama, A. (1997).

Reassessment of FDG uptake in tumor cells: high FDG uptake as a reflection of oxygen-independent glycolysis dominant energy production. Nucl Med Biol, 24, 665-70.
89. Waki, A., Kato, H., Yano, R., Sadato, N., Yokoyama, A., Ishii, Y., Yonekura, Y. & Fujibayashi, Y. (1998). The importance of glucose transport activity as the rate-limiting step of 2-deoxyglucose uptake in tumor cells in vitro. Nucl Med Biol, 25, 593-7.

90. Warburg, O. H. (1956). Science, 123, 309-314.
91. Waschsberger, P. R., Gressen, E. L., Bhala, A. & al, e. (2002). Variability in glucose transporter-1 levels and hexokinase activity in human melanoma. Melanoma Res, 12, 35-43.
92. Wood, L, Leese, M. P., Mouzakiti, A., Purohit, A., Potter, B. V., Reed, M. J. & Packham, G. (2004). 2-MeOE2bisMATE induces caspase-dependent apoptosis in CAL51 breast cancer cells and overcomes resistance to TRAIL via cooperative activation of caspases. Apoptosis, 9, 323-32.
93. Xiang, X., Saha, A. K., Wen, R., Ruderman, N. B. & Luo, Z. (2004). AMP-activated protein kinase activators can inhibit the growth of prostate cancer cells by multiple mechanisms. Biochem Biophys Res Commun, 321, 161-167.
94. Xu, R.-H., Pelicano, H., Zhou, Y., Carew, J. S., Feng, L., Bhalla, K. N., Keating, M. J. & Huang, P. (2005). Inhibition of Glycolysis in Cancer Cells: A Novel Strategy to Overcome Drug Resistance Associated with Mitochondrial Respiratory Defect and Hypoxia. Cancer Res, 65, 613-621.
95. Yue, T. L., Wang, X., Louden, C. S. & al, e. (1997). 2-Methoxyestradiol, an endogenous estrogen metabolite, induces apoptosis in endothelial cells and inhibits angiogenesis: Possible role for stress-activated protein kinase,signaling pathway and Fas expression. Mol Pharmacol., 51, 951-962.
96. Zhou, Y., Hileman, E. O., Plunkett, W., Keating, M. J. & Huang, P. (2003). Free radical stress in chornic lymphocytic leukaemia cells and its role in cellular sensitivity to ROS-generating anticancer agents. Blood., 101, 4098-4104.
97. Zhu, Z., Jiang, W., McGinley, J. N. & Thompson, H. J. (2005). 2-Deoxyglucose as an Energy Restriction Mimetic Agent: EFfects on Mammary Carcinogenesis and on Mammary Tumor Cell Growth In vitro. Cancer Res, 65, 7023.
98. Zong, W. X., Edelstein, L. C., Chen, C., Bash, J. & Gelinas, C. (1999). The prosurvival Bcl-2 homolog Bfl-1/A1 is a direct transcriptional target of NF-kappaB that blocks TNFalpha-induced apoptosis. Genes & Development, 13, 382-7.

1. A composition comprising:
(a) a glycolytic inhibitor; and
(b) a compound comprising a ring system substituted with at least one of a sulphamate group and an alkoxy group;
wherein (a) and (b) are different.

2. (canceled)

3. The composition according to claim 1, wherein the glycolytic inhibitor is a glucose analogue or a glucose conjugate.

4. The composition according to claim 1, wherein the glycolytic inhibitor is a compound of the formula: 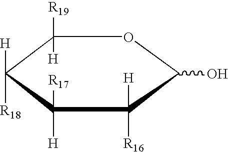
wherein each of R₁₆, R₁₇, R₁₈ is independently H, OH, OSO₂NH₂, OSO₃H, SO₃H, oxamate or halogen and wherein R₁₉ is CH₂OH.

5. The composition according to claim 1, wherein the glycolytic inhibitor is a compound of the formula: 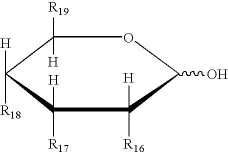
wherein each of R₁₆, R₁₇, R₁₈ is independently H, OH, OSO₂NH₂, OSO₃H, SO₃H, oxamate or halogen and wherein R₁₉ is CH₃.

6. The composition according to one of claim 4, wherein R₁₆ is H, R₁₇ is OH, and R₁₈ is OH.

7. The composition according to claim 1, wherein the glycolytic inhibitor is 2-deoxy-D-glucose, 1,6-dichloro-1,6-dideoxy-2-deoxyglucose, 2-[N-(7-nitrobenz-2-oxa-1,3-diaxol-4-yl)amino]-2-deoxyglucose (2-NBDG), 2-fluor-2-deoxy-D-glucose (2FG), 2-deoxy-D-galactose, 3H-2-deoxyglucose or mixtures thereof.

8. The composition according to claim 1 wherein the glycolytic inhibitor is 2-deoxy-D-glucose.

9. The composition according to claim 1, wherein the compound comprises a ring system substituted with a sulphamate group and an alkoxy group.

10. The composition according to claim 1, wherein the ring system is a steroidal ring system.

11. The composition according to claim 1, wherein the compound comprises a steroidal ring system substituted with a sulphamate group and an alkoxy group.

12. The composition according to claim 1, wherein the compound is of Formula I: 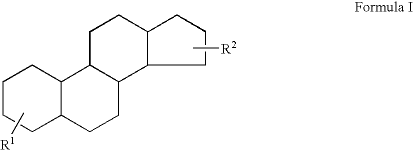
wherein R¹ is —OH or a sulphamate group;
wherein R² is —OH, a sulphamate group, ═O or L-R³, wherein L is an optional linker group and R³ is:
(i) —SO₂R⁵, wherein R⁵ is H, a hydrocarbyl group or a bond or group attached to the D ring;
(ii) —NO₂;
(iii) —SOR⁶, wherein R⁶ is H or a hydrocarbyl group;
(iv) —R⁷, wherein R⁷ is a halogen;
(v) -alkyl;
vi) —C(═O)R⁸, wherein R⁸ is H or hydrocarbyl;
(vii) —C═CR⁹, wherein R⁹ is H or hydrocarbyl;
(viii) —OC(═O)NR¹⁰R¹¹, wherein R¹⁰ and R¹¹ are independently H or hydrocarbyl; 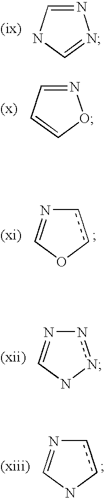
(xiv) a nitrile group;
(xv) an alcohol;
(xvi) an ester;
(xvii) an ether;
(xviii) an amine; or
(xix) an alkene.

13. The composition according to claim 1, wherein the compound is of Formula: 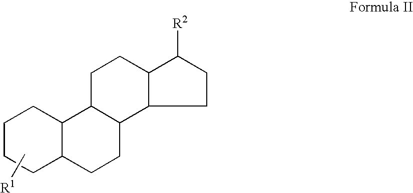
wherein R¹ is —OH or a sulphamate group;
wherein R² is —OH, a sulphamate group, ═O or L-R³, wherein L is an optional linker group and R³ is:
(i) —SO₂R⁵, wherein R⁵ is H, a hydrocarbyl group or a bond or group attached to the D ring;
ii —NO₂;
(iii) —SOR⁶, wherein R⁶ is H or a hydrocarbyl group;
(iv) —R⁷, wherein R⁷ is a halogen;
(v) -alkyl;
(vi) —C(⊚O)R⁸, wherein R⁸ is H or hydrocarbyl;
(vii) —C═CR⁹, wherein R⁹ is H or hydrocarbyl;
(viii) —OC(═O)NR¹⁰R¹¹, wherein R¹⁰ and R¹¹ are independently H or hydrocarbyl; 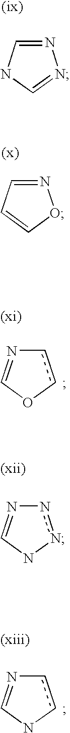
(xiv) a nitrile group;
(xv) an alcohol;
(xvi) an ester;
(xvii) an ether;
(xviii) an amine; or
(xix) an alkene.

14. The composition according to claim 1, wherein the compound is of Formula III: 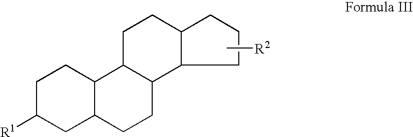
wherein R¹ is —OH or a sulphamate group;
wherein R² is —OH, a sulphamate group, ═O or L-R³, wherein L is an optional linker group and R³ is:
(i) —SO₂R⁵, wherein R⁵ is H, a hydrocarbyl group or a bond or group attached to the D ring;
ii) —NO₂;
(iii) —SOR⁶, wherein R⁶ is H or a hydrocarbyl group;
(iv) —R⁷, wherein R⁷ is a halogen;
(v) -alkyl;
(vi) —C(═O)R⁸, wherein R⁸ is H or hydrocarbyl;
(vii) —C═CR⁹, wherein R⁹ is H or hydrocarbyl;
(viii) —OC(═O)NR¹⁰R¹¹, wherein R¹⁰ and R¹¹ are independently H or hydrocarbyl; 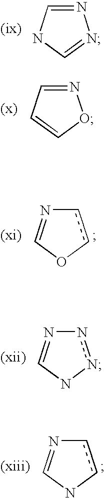
(xiv) a nitrile group;
(xv) an alcohol;
(xvi) an ester;
(xvii) an ether;
(xviii) an amine; or (xix) an alkene.

15. The composition according to claim 1, wherein the compound is of Formula IVa or Formula IVb: 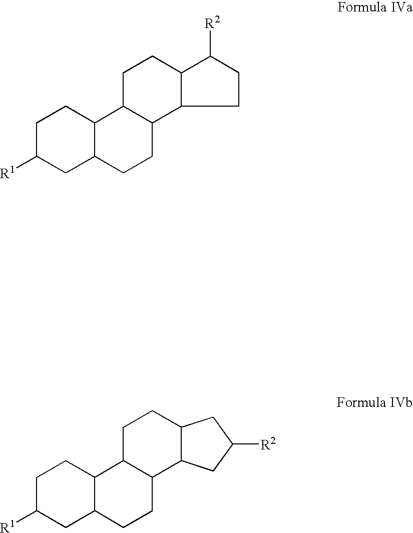
wherein R¹ is —OH or a sulphamate group;
wherein R² is —OH, a sulphamate group, ═O or L-R³, wherein L is an optional linker group and R³ is:
(i) —SO₂R⁵, wherein R⁵ is H, a hydrocarbyl group or a bond or group attached to the D ring;
(ii) —NO₂;
(iii) —SOR⁶, wherein R⁶ is H or a hydrocarbyl group;
(iv) —R⁷, wherein R⁷ is a halogen;
(v) -alkyl;
(vi) —C(═O)R⁸, wherein R⁸ is H or hydrocarbyl;
(vii) —C═CR⁹, wherein R⁹ is H or hydrocarbyl;
(viii) —OC(═O)NR¹⁰R¹¹, wherein R¹⁰ and R¹¹ are independently H or hydrocarbyl; 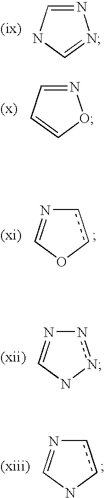
(xiv) a nitrile group;
(xv) an alcohol;
(xvi) an ester;
(xvii) an ether;
(xviii) an amine; or
(xix) an alkene.

16. The composition according to claim 1, wherein the compound is of Formula IVc: 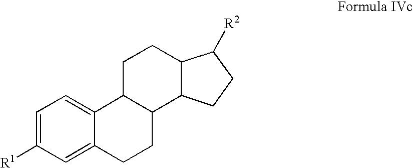
wherein R¹ is —OH or a sulphamate group;
wherein R² is —OH, a sulphamate group, ═O or L-R³, wherein L is an optional linker group and R³ is:
(i) —SOR⁵, wherein R⁵ is H, a hydrocarbyl group or a bond or group attached to the D ring;
(ii) —NO₂;
(iii) —SOR⁶, wherein R⁶ is H or a hydrocarbyl group;
(iv) —R⁷, wherein R⁷ is a halogen;
(v) -alkyl;
(vi) —C(═O)R⁸, wherein R⁸ is H or hydrocarbyl;
(vii) —C═CR⁹, wherein R⁹ is H or hydrocarbyl;
(viii) —OC(═O)NR¹⁰R¹¹, wherein R¹⁰ and R¹¹ are independently H or hydrocarbyl; 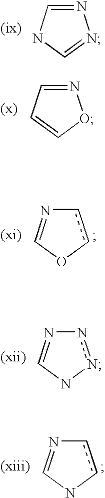
(xiv) a nitrile group;
(xv) an alcohol;
(xvi) an ester;
(xvii) an ether;
(xviii) an amine; or
(xix) an alkene.

17. The composition according to claim 12, wherein the A ring of the steroidal ring system further comprises a group R⁴ at position 2 or 4 of the steroidal ring⁴, wherein R⁴ is a hydrocarbyl group or an oxyhydrocarbyl group.

18. The composition according to claim 17, wherein R⁴ is an oxyhydrocarbyl group.

19. The composition according to claim 18, wherein R⁴ is an alkoxy group.

20. The composition according to claim 19, wherein R⁴ is methoxy.

21. The composition according to claim 17, wherein R⁴ is a hydrocarbyl group.

22. The composition according to claim 21, wherein R⁴ is an alkyl group.

23. The composition according to claim 22, wherein R⁴ is ethyl.

24. The composition according to claim 17, wherein R⁴ is at position 2 of the A ring.

25. The composition according to claim 12, wherein R¹ is —OH or a sulphamate group.

26. The composition according to claim 12, wherein R¹ is —OH.

27. The composition according to claim 12, wherein R¹ is a sulphamate group.

28. The composition according to claim 27, wherein R¹ is a sulphamate group of the formula: 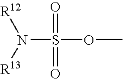
wherein R¹² and R¹³ are independently H, alkyl, cycloalkyl, alkenyl, aryl, or combinations thereof, or together represent alkylene, wherein each alkyl or cycloalkyl or alkenyl or aryl optionally contains one or more hetero atoms or groups.

29. The composition according to claim 28, wherein at least one of R¹² and R¹³ is H.

30. The composition according to claim 28, wherein each of R¹² and R¹³ is H.

31. The composition according to claim 12, wherein L is a hydrocarbyl group, —NR¹⁴— or —O—, wherein R¹⁴ is H, a hydrocarbyl group or a bond.

32. The composition according to claim 31, wherein L is hydrocarbyl group, —NR¹⁴— and or —O—.

33. The composition according to claim 31, wherein L is an alkylene group, —NR¹⁴— or —O—.

34. The composition according to claim 31, wherein L is a C₁₋₁₀ alkylene group, —NR¹⁴— or —O—.

35. The composition according to claim 31, wherein L is a C₁ or C₂ alkylene group, —NR¹⁴— or —O—.

36. The composition according to claim 12, wherein R² is —O— or a sulphamate group.

37. The composition according to claim 12, wherein R² is a sulphamate group.

38. The composition according to claim 12, wherein R² is a sulphamate group of the formula: 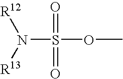
wherein R¹² and R¹³ are independently H, alkyl, cycloalkyl, alkenyl, aryl, or combinations thereof, or together represent alkylene, wherein the or each alkyl or cycloalkyl or alkenyl or aryl optionally contains one or more hetero atoms or groups.

39. The composition according to claim 12, wherein group R² is in an α configuration.

40. The composition according to claim 1, wherein the compound is of Formula Va: 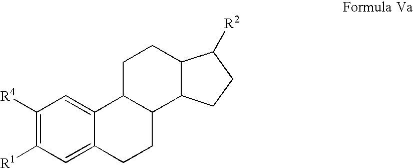
wherein R¹ is —OH or a sulphamate group:
wherein R² is —OH or a sulphamate group;
wherein each sulphamate group is of the formula: 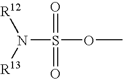
wherein R¹² and R¹³ are independently H or alkyl;
and wherein R⁴ is an alkoxy group.

41. The composition according to claim 1, wherein the compound is:

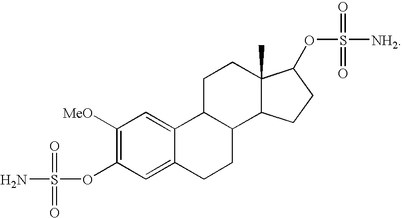

42. A pharmaceutical composition comprising:
(a) a composition as defined in claim 1, and
(b) a pharmaceutically acceptable carrier, diluent, excipient or adjuvant.

43. (canceled)

44. A method of preventing and/or inhibiting tumor growth comprising administering to a subject in need thereof a therapeutically effective amount of the composition of claim 1.

45. A method of treating a condition or disease associated with one or more of steroid sulphatase (STS) activity; cell cycling;
apoptosis; cell growth; glucose uptake by a tumor; tumor angiogenesis; microtubules formation; and apoptosis comprising administering to a subject in need thereof a therapeutically effective amount of a composition of claim 1.

46. A method of treating a condition or disease associated with adverse levels of one or more of steroid sulphatase (STS) activity; cell cycling; apoptosis; cell growth; glucose uptake by a tumor; tumor angiogenesis; microtubules formation; and apoptosis comprising administering to a subject in need thereof a therapeutically effective amount of a composition of claim 1.

47. A method of inhibiting steroid sulphatase (STS) activity; modulating cell cycling; modulating apoptosis; modulating cell growth;
preventing and/or suppressing glucose uptake by a tumor; preventing and/or inhibiting tumor angiogenesis; disrupting microtubules; or inducing apoptosis comprising administering to a subject in need thereof a therapeutically effective amount of a composition of claim 1.

48. A method treating a cancer comprising administering to a subject in need thereof a therapeutically effective amount of a composition of claim 1.

49. The method according to claim 48, wherein the cancer is a solid tumor.

50. The method according to claim 48, wherein the cancer is breast cancer, ovarian cancer or prostate cancer.

51. A method of rendering a tumor susceptible to action by a glycolytic inhibitor comprising administering to a subject in need thereof a therapeutically effective amount of a compound comprising a ring system substituted with at least one of a sulphamate group or an alkoxy group.

52. The method according to claim 51, wherein the compound comprises a ring system substituted with a sulphamate group and an alkoxy group and/or the glycolytic inhibitor in a glucose analogue or a glucose conjugate.

53. A method of intensifying at least one of hypoxia and glycolysis in a tumor comprising administering to a subject in need thereof a therapeutically effective amount of a compound comprising a ring system substituted with at least one of a sulphamate group and an alkoxy group.

54. (canceled)

55. A method for decreasing ATP levels in a tumor comprising administering to a subject in need thereof a therapeutically effective amount of a composition of claim 1.

56. (canceled)

57. (canceled)

58. (canceled)

59. (canceled)

60. (canceled)
